(19) United States
(12) Patent Application Publication
(10) Pub. No.: US 2025/0058031 A1
(43) Pub. Date: Feb. 20, 2025

(54) SYSTEM AND METHOD FOR PRODUCING DIALYSIS FLUID

(71) Applicant: GAMBRO LUNDIA AB, Lund (SE)

(72) Inventors: Markus NILSSON, Lund (SE); Henrik LINDGREN, Genarp (SE); Christina SCRET, Dalby (SE); Tarakranjan GUPTA, Horamavu, Bangalore (IN); Christian VARTIA, Veberöd (SE)

(21) Appl. No.: 18/723,744

(22) PCT Filed: Dec. 20, 2022
(86) PCT No.: PCT/EP2022/086927

§ 371 Date: Jun. 24, 2024

(30) Foreign Application Priority Data
Dec. 22, 2021 (IN) 202141059868
Feb. 15, 2022 (SE) 2250148-0

(51) Int. Cl. A61M 1/16; B01D 61/00; C02F 1/00; C02F 1/44

(52) U.S. Cl. CPC A61M 1/1696; A61M 1/1609; A61M 1/1672; B01D 61/0022; B01D 61/0024; C02F 1/008; C02F 1/445; A61M 2205/3327; A61M 2205/3606

(57) ABSTRACT

A system for producing dialysis fluid comprises: a forward osmosis, FO, unit comprising a feed side and a draw side separated by an FO membrane, a first subsystem for providing spent fluid to the feed side, a second fluid sub-system for providing a concentrate fluid to the draw side, and a third sub-system for receiving a diluted concentrate fluid from the draw side and processing the diluted concentrate fluid into a final dialysis fluid. A water supply unit is configured to extract liquid water from ambient air. The water supply unit is fluidly connected to provide process water, which includes the extracted liquid water, to at least one of (i) the first fluid sub-system for combination with the spent fluid, (ii) the second fluid subsystem for admixing into the concentrate fluid, or (iii) the third sub-system.

### TECHNICAL FIELD

The present disclosure relates generally to dialysis therapy, and in particular to a technique of producing dialysis fluid for use in dialysis therapy.

### BACKGROUND ART

In the treatment of individuals suffering from acute or chronic renal insufficiency, dialysis therapy may be needed.

One type of dialysis therapy is extracorporeal (EC) blood therapy, in which blood from a patient is pumped through an EC blood circuit back to the patient. A blood filtration unit, commonly known as a dialyzer, is arranged in the EC blood circuit to interface the blood with a dialysis fluid over a semi-permeable membrane. One modality of EC blood therapy is hemodialysis (HD) which in general uses diffusion to remove waste products from the blood. A diffusive gradient occurs across the semi-permeable membrane and the dialysis fluid. Another modality is hemofiltration (HF), which relies on convective transport of toxins from the patient's blood. HF is accomplished by adding another dialysis fluid, denoted substitution or replacement fluid, to the extracorporeal blood circuit during dialysis therapy. The replacement fluid and excess fluid accumulated by the patient in between therapy sessions is ultrafiltered over the course of HF therapy, providing a convective transport mechanism that is particularly beneficial in removing middle and large molecules. Yet another modality is hemodiafiltration (HDF), which combines convective and diffusive clearances. HDF uses dialysis fluid flowing through a dialyzer, similar to standard hemodialysis, to provide diffusive clearance. In addition, replacement fluid is delivered directly to the extracorporeal blood circuit, providing convective clearance. Here, more fluid than the patient's excess fluid is removed from the blood, causing the increased convective transport of waste products from the blood. The additional fluid removed is replaced via the replacement fluid.

Another type of dialysis therapy is peritoneal dialysis (PD), in which a dialysis fluid is infused into a patient's peritoneal cavity. The dialysis fluid is in contact with the peritoneal membrane located in the patient's peritoneal cavity. Waste, toxins and excess water pass from the patient's bloodstream, through the capillaries in the peritoneal membrane, and into the dialysis fluid by diffusion and osmosis, i.e., an osmotic gradient occurs across the membrane. An osmotic agent in the dialysis fluid provides the osmotic gradient. Used or spent dialysis fluid is drained from the patient, removing waste, toxins and excess water from the patient. This cycle is repeated, for example multiple times.

There are various types of PD therapies, including continuous ambulatory PD (CAPD), automated PD (APD), tidal PD (TPD), and continuous flow PD (CFPD). CAPD is a manual dialysis treatment, in which the flow of treatment fluid into and out of the patient is driven by gravity. APD is performed by a dialysis machine, commonly known as a cycler, which is fluidly connected to the peritoneal cavity and operated to automatically transfer treatment fluid to and from the peritoneal cavity in accordance with a predefined schedule, for example during the night while the patient is sleeping.

Conventionally, dialysis fluid for PD is delivered in pre-filled bags to the point-of-care, for example an intensive care unit or the home of the patient. EC blood therapy may also use pre-filled bags of dialysis fluid, for example in an intensive care unit, for treatment of acute kidney failure. Dialysis fluid for EC blood treatment of chronic kidney failure is typically produced by the dialysis machine itself, by mixing one or more concentrates with purified water. Recently, dialysis machines that produce dialysis fluid for PD have also been proposed.

Local production of dialysis fluid at the point-of-care is attractive since it reduces the cost and environmental impact of transporting large amounts of ready-made dialysis fluid and the burden of storing and handling pre-filled bags. However, production of dialysis fluid requires access to purified water. Typically, a water purifier is connected to a tap water source, and a fluid generation unit is operated to mix one or more concentrates with the purified water to generate the dialysis fluid. Large amounts of tap water may be consumed. In PD, about 15 liters of dialysis fluid is consumed during each therapy session. In EC blood therapy, more than 100 liters of dialysis fluid may be consumed during a single therapy session. Correspondingly, large amounts of spent dialysis fluid is produced in dialysis therapy. The spent dialysis fluid may be directed to a drain at the point-of-care.

There is a general need to reduce the consumption of water during dialysis therapy.

There is also a general need to facilitate installation of a dialysis system that is configured to produce dialysis fluid. At present, the need for tap water and the need to dispose of the spent dialysis fluid restrict the installation. The tap water source and the drain may be located far away from the desired location of the dialysis system, requiring significant plumbing work and the use of extended tubing, which in turn increases the risk for leaks and consequential water damage. U.S. Pat. No. 10,632,242 discloses a dialysis machine that is designed to operate in areas where resources such as energy and clean water are scarce. The dialysis machine comprises a condenser-based water generator which is powered by a solar panel to extract water from ambient air. After passing an ultrafilter, the extracted water may be mixed with concentrates to form a dialysis fluid. The dialysis fluid is added to regenerated dialysis fluid, which is produced from spent dialysis fluid by use of a sorbent device in a conventional regeneration circuit. The extracted water may alternatively be input to a forward osmosis container that is configured to allow water to mix with a salt concentrate to produce a saline solution to be infused into the patient's blood.

### SUMMARY

It is an objective to at least partly overcome one or more limitations of the prior art.

One objective is to provide a cost-effective technique for producing dialysis fluid at the point-of-care.

Another objective is to provide a technique for reducing the consumption of water during dialysis therapy.

Yet another objective is to mitigate the need to dispose of spent dialysis fluid that is generated in dialysis therapy.

One or more of these objectives, as well as further objectives that may appear from the description below, are at least partly achieved by a system and a method for producing dialysis fluid, embodiments thereof being defined by the dependent claims.

A first aspect is a system for producing dialysis fluid based on spent fluid. The system comprises: a forward osmosis, FO, unit comprising a feed side and a draw side separated by an FO membrane, the FO unit being arranged to receive the spent fluid at an inlet on the feed side and receive a concentrate fluid at an inlet on the draw side, and being configured to transport water from the spent fluid to the concentrate fluid through the FO membrane via an osmotic pressure gradient between the feed side and the draw side, thereby diluting the concentrate fluid into a diluted concentrate fluid. The system further comprises: a first sub-system fluidly connected to provide the spent fluid to the inlet on the feed side of the FO unit; a second fluid sub-system fluidly connected to provide the concentrate fluid to the inlet on the draw side of the FO unit; a third sub-system fluidly connected to receive the diluted concentrate fluid from an outlet on the draw side of the FO unit, the third sub-system being configured to process the diluted concentrate fluid into a final dialysis fluid; and a water supply unit which is configured to extract liquid water from ambient air and provide process water that includes the extracted liquid water. The water supply unit is fluidly connected to provide the process water to at least one of (i) the first fluid sub-system for combination with the spent fluid, (ii) the second fluid sub-system for admixing into the concentrate fluid, or (iii) the third sub-system for use in processing the diluted concentrate fluid into the final dialysis fluid.

The system of the first aspect combines water extraction from air with a forward osmosis (FO) unit to produce dialysis fluid at minimum consumption of fresh water or tap water. The FO unit is operated to extract water from spent fluid, by directly driving water from the spent fluid into a concentrate fluid, which is to be part of the final dialysis fluid to be generated. The output of the forward osmosis unit is thus a diluted version of the concentrate fluid. The water extracted from ambient air by the water supply unit is added to the system to supplement the dilution performed in the FO unit. Depending on desired system performance and configuration, the water extracted from ambient air may be added in the system either upstream or downstream of the FO unit, or both. By combining the extracted water with the spent fluid upstream of the FO unit, in the first sub-system, the FO membrane may perform an inherent purification and/or sterilization of the extracted water when driven into the concentrate fluid. The inherent purification and/or sterilization may reduce the requirements on the water supply unit, as well as reduce the overall cost and complexity of the system. Further, the osmotic pressure gradient in the FO unit may be increased due to an increased difference in concentration between the feed and draw sides by the presence of the extracted water on the feed side. By adding the extracted water to the diluted concentrate fluid, in the third sub-system downstream of the FO unit, system control may be facilitated. For example, the diluted concentrate fluid may be processed in correspondence with conventional practice, in which a concentrate is mixed with water and possibly one or more additional concentrates to attain a specific composition of the final dialysis fluid. Another possibility is to add the extracted water to the concentrate fluid in the second sub-system upstream of the FO unit.

Before being added in the system, the extracted water may be collected in a container within the system. Part of the extracted water in the container may be diverted for other uses in relation to the system, for example to be used in priming or sanitization of the first to third sub-systems or any part thereof.

The water supply unit need not provide only liquid water that is extracted from ambient air. Hence, the “process water” provided by the water supply unit may include water from other sources as well, for example tap water.

The spent fluid that is processed in the FO unit comprises spent dialysis fluid from a therapy system. Depending on implementation, the spent fluid may also include other types of spent fluid as produced in dialysis therapy, such as priming fluid, cleaning fluid, etc.

The use of forward osmosis will significantly reduce the consumption of water compared to systems that direct the spent fluid to drain, since at least part of the water in the spent fluid is recuperated (recovered) in the FO unit. By combining forward osmosis with water extraction from ambient air, it is possible to reduce the consumption of tap water to a minimum, or even eliminate the need for tap water altogether. Further, the water that is extracted from ambient air will allow the FO unit to be operated at less than its maximum capacity, since the liquid water from air is available to supplement the water drawn by the FO unit from the spent fluid. This may, in turn, result in a longer life of the FO unit, higher flow rate of the concentrate fluid through the FO unit, 10 reduced risk for operative failure, etc. The combination of forward osmosis and water extraction from ambient air also enables cost-effective production of dialysis fluid, for example compared to conventional regeneration of dialysis fluid. Conventional regeneration is reliant on relatively costly sorbent cartridges, which comprise stacked layers of different sorbents tailored to adsorb a respective substance in spent dialysis 15 fluid as it passes the stacked layers. Sorbent cartridges are also associated with other drawbacks such as low flow rates of dialysis fluid, leaching of harmful substances from the sorbents, incomplete regeneration of dialysis fluid, etc. By contrast, the combination of forward osmosis and water extraction from ambient air enables production of dialysis fluid with a well-defined composition, by careful design and operation of at least the FO unit and the third sub-system. Further, by reducing the water content of the spent fluid, the FO unit will also inherently reduce the amount of spent fluid that needs to be handled after a session of dialysis therapy.

In some embodiments, the water supply unit comprises a desiccant, which is arranged to adsorb and/or absorb moisture from an incoming stream of ambient air and which is processed by the water supply unit to extract the liquid water from the desiccant.

In some embodiments, the desiccant is configured to have a high selectivity towards water.

In some embodiments, the liquid water that is extracted from the ambient air has conductivity of less than 10 μS/cm, and preferably less than 5 μS/cm or 1 μS/cm.

In some embodiments, the water supply unit comprises a cooling element, which is configured to cool an incoming stream of ambient air to extract the liquid water from the incoming stream of ambient air by condensation.

In some embodiments, the process water consists of the extracted liquid water.

In some embodiments, the system is arranged to provide the final dialysis fluid to a therapy system, which is configured for performing dialysis therapy, and receive the spent fluid from the therapy system, the spent fluid comprising spent dialysis fluid generated by the therapy system as a result of the dialysis therapy.

In some embodiments, the system further comprising a control arrangement, which is configured to jointly operate the water supply unit and the FO unit to achieve a target production rate of the final dialysis fluid.

In some embodiments, the target production rate is adapted to a consumption rate of the final dialysis fluid during a current treatment session of the dialysis therapy.

In some embodiments, the control arrangement is configured to estimate a water consumption rate for the current treatment session, and determine an estimated amount of the process water that is produced by the water supply unit between treatment sessions, and optionally during the current treatment session, and configure the FO unit for operation during the current treatment session in dependence of the estimated water consumption rate and the estimated amount of the process water.

In some embodiments, the FO unit has a maximum water extraction capacity, and wherein the control arrangement is configured, in view of the estimated amount of the process water, to operate the FO unit with a water extraction capacity below the maximum water extraction capacity during the current treatment session.

In some embodiments, the control arrangement is configured to operate the water supply unit to produce the estimated amount of the process water so that the FO unit is operable at a fraction, a, of the maximum water extraction capacity, wherein α≤0.95 and preferably α≤0.9.

In some embodiments, the control arrangement is configured to operate the FO unit to produce less than 90% of an estimated total consumption of the process water during the current treatment session.

In some embodiments, the control arrangement is configured to operate the water supply unit to maximize extraction of the liquid water from the ambient air, at least between treatment sessions, while maintaining a humidity of the ambient air above a humidity limit.

In some embodiments, the process water that is produced by the water supply unit is stored in a PW container, which is fluidly connected to at least one of the first sub-system, the second sub-system or the third sub-system.

In some embodiments, each treatment session comprises a series of fluid exchange cycles, wherein each of the fluid exchange cycles comprises a fill phase, in which a first amount of the final dialysis fluid is supplied to a peritoneal cavity of a patient, a dwell phase, in which the final dialysis fluid resides in the peritoneal cavity, and a drain phase, in which a second amount of the spent fluid is withdrawn from the peritoneal cavity, and the PW container is fluidly connected to the third fluid sub-system to provide the processing water for use in processing the diluted concentrate fluid into the final dialysis fluid.

In some embodiments, the control arrangement is configured to operate the FO unit to produce a third amount of the diluted concentrate fluid from the second amount of the spent fluid withdrawn in a respective drain phase, and operate the third sub-system to generate the first amount of the final dialysis fluid for use in a fill phase, which is subsequent to the respective drain phase, based on the third amount of the diluted concentrate fluid and a supplementary amount of the process water in the PW container.

In some embodiments, the control arrangement is configured to operate the water supply unit, at least between treatment sessions, to generate and accumulate process water in the PW container, and the control arrangement is configured to set a target value for the third amount based on a fourth amount of process water in the PW container at start of the current treatment session.

In some embodiments, the PW container is a disposable unit.

In some embodiments, the PW container defines a fluid compartment in contact with the process water, the fluid compartment being lined by an antibacterial material.

In some embodiments, the PW container is associated with a sterilization device, which is operable to sterilize the PW container and/or the process water.

In some embodiments, the water supply unit is fluidly connected to provide the process water to at least one of the second fluid sub-system or the third fluid sub-system, the system further comprising at least one sterilization unit, which is arranged to sterilize at least one of the process water, the concentrate fluid, the diluted concentrate fluid or the final dialysis fluid.

In some embodiments, the system comprises a sensor arrangement, which is configured to generate sensor data representative of spatial structures around the water supply unit, and is configured to process the sensor data to estimate an available volume of ambient air and operate the water supply unit based on the available volume of ambient air.

In some embodiments, the first sub-system is configured to admix the process water into the spent fluid that is provided to the inlet on the feed side of the FO unit.

In some embodiments, the first sub-system is configured to alternately provide the spent fluid and the process water to the inlet on the feed side of the FO unit.

In some embodiments, the system further comprises an SF container, which is arranged for intermediate storage of the spent fluid and is fluidly connected to the inlet on the feed side of the FO unit.

In some embodiments, the SF container is associated with a sterilization device which is operable to sterilize the SF container and/or the spent fluid.

In some embodiments, the second sub-system is configured to generate the concentrate fluid to include one or more liquid dialysis concentrates.

In some embodiments, the second sub-system is configured to dilute the one or more liquid dialysis concentrates by the process water to form the concentrate fluid.

In some embodiments, the third sub-system comprises at least one mixing section for mixing the diluted concentrate fluid with process water and/or at least one dialysis concentrate.

In some embodiments, the third sub-system is configured to first mix the diluted concentrate fluid with the process water to generate a fluid mixture and then mix the fluid mixture with said at least one dialysis concentrate.

In some embodiments, the third sub-system comprises a first sensor for measuring a first concentration of the diluted concentrate fluid and second sensor for measuring a second concentration of the final dialysis fluid, and the system is operable to control, based on the first and second concentrations, a flow rate of the diluted concentrate fluid, and one or more flow rates of the process water and/or said at least one dialysis concentrate.

In some embodiments, the diluted concentrate fluid comprises electrolytes of the final dialysis fluid, and said at least one dialysis concentrate comprises at least one of an osmotic agent or a buffer of the final dialysis fluid.

In some embodiments, the final dialysis fluid is a dialysis fluid for use in peritoneal dialysis, or a dialysis fluid or replacement fluid for use in extracorporeal blood therapy.

A second aspect is a method of producing dialysis fluid based on spent fluid. The method comprises: supplying spent fluid to an inlet on a feed side of a forward osmosis, FO, unit; and supplying a concentrate fluid to an inlet on a draw side of the FO unit, the draw side being separated from the feed side by an FO membrane, the FO unit being configured to transport water from the spent fluid to the concentrate fluid through the FO membrane via an osmotic pressure gradient between the feed side and the draw side, thereby diluting the concentrate fluid into a diluted concentrate fluid. The method further comprises: obtaining the diluted concentrate fluid from an outlet on the draw side of the FO unit; and processing the diluted concentrate fluid into a final dialysis fluid. The method further comprises: extracting liquid water from ambient air; and supplying process water, which includes the extracted liquid water, for use in producing the final dialysis fluid, wherein said supplying the process water comprises at least one of (i) supplying the process water in combination with the spent fluid to the inlet on the feed side of the FO unit, (ii) supplying the process water for admixing into the concentrate fluid, or (iii) supplying the process water for use in said processing the diluted concentrate fluid into the final dialysis fluid.

The embodiments of the first aspect may be adapted as embodiments of the second aspect.

A third aspect is a computer-readable medium comprising program instructions, which when executed by a processor causes the processor to perform the method of the second aspect, or any embodiment thereof. The computer-readable medium may be a non-transitory medium or a propagating signal.

Still other objectives, aspects, embodiments, and technical effects, as well as features and advantages may appear from the following detailed description, from the attached claims as well as from the drawings.

### BRIEF DESCRIPTION OF THE DRAWINGS

Embodiments will now be described in more detail with reference to the accompanying and schematic drawings.

FIGS. 1A-1C are schematic views of example systems for dialysis therapy.

FIG. 2 is a block diagram of an example dehumidifier unit for extraction of liquid water from air.

FIGS. 3A-3C are block diagrams of example systems for producing dialysis fluid from spent dialysis fluid.

FIGS. 4A-4C are flow charts of example operating methods for the systems in FIGS. 3A-3C.

FIG. 5A is a block diagram of an example third sub-system in the system of FIG. 3B, and FIGS. 5B-5D are section views of example containers for storing process water in the systems of FIGS. 3A-3C.

FIG. 6A is a circuit diagram of an example system for producing dialysis fluid for peritoneal dialysis (PD), FIG. 6B is a flow chart of an example method of operating the system in FIG. 6A, FIGS. 6C-6D show examples of fluid exchange cycles during PD, and FIGS. 6E-6G show examples of water production and water consumption in relation to PD.

FIG. 7 is a flow chart of a method of producing dialysis fluid.

FIG. 8 is a block diagram of an example system for producing dialysis fluid according to a variant.

### LIST OF ABBREVIATIONS

APD Automated peritoneal dialysis
AW Auxiliary water
CAPD Continuous ambulatory peritoneal dialysis
Ci Dialysis concentrate
CF Concentrate fluid
CPD Continuous flow peritoneal dialysis
Ce Dialysis concentrate
DCF Diluted concentrate fluid
DHU Dehumidifier unit
DIA Incoming air stream (dehumidifier unit)
DOA Outgoing air stream (dehumidifier unit)
EC Extracorporeal
EW Extracted liquid water
FDF Final concentrate fluid
FGU Fluid generation unit
FO Forward osmosis
FPS Fluid preparation system
HD Hemodialysis
Hdi Inlet humidity (dehumidifier unit)
HDF Hemodiafiltration
Hdo Outlet humidity (dehumidifier unit)
HF Hemofiltration
MOF Metal-organic framework
PC Peritoneal cavity
PD Peritoneal dialysis
PW Process water
RF Residual fluid
RH Relative humidity
SBU Secondary building unit
SF Spent fluid
TPD Tidal peritoneal dialysis
TW Tap water
WSU Water supply unit

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

As used herein, the terms “multiple”, “plural” and “plurality” are intended to imply provision of two or more elements, whereas the term a “set” of elements is intended to imply a provision of one or more elements. The term “and/or” includes any and all combinations of one or more of the associated listed elements.

It will furthermore be understood that although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure.

Well-known functions or constructions may not be described in detail for brevity and/or clarity. Unless otherwise defined, all terms (including technical and scientific terms) used herein have the same meaning as commonly understood by one of ordinary skill in the art to which this disclosure belongs.

As used herein, “dialysis therapy” refers to any therapy that replaces or supplements the renal function of a patient by use of dialysis fluid. Dialysis therapy includes, without limitation, extracorporcal blood therapy and peritoncal dialysis therapy.

As used herein, “final dialysis fluid” refers to any fluid that is consumed as a result of dialysis therapy. Dialysis fluid includes, without limitation, fluid for infusion into the peritoneal cavity during peritoneal dialysis therapy, fluid for supply to a dialyzer during EC blood therapy, and replacement fluid and substitution fluid for infusion into blood during EC blood therapy.

As used herein, “sterilization” refers to any process that substantially removes, kills, or deactivates microorganisms and other biological agents. In the context of the present disclosure, no distinction is made between sterilization, disinfection, and sanitization. Sterilization may involve applying one or more of heat, chemicals, irradiation, high pressure, or filtration.

As used herein, “purification” refers to a process of substantially removing undesirable chemicals, biological contaminants, suspended solids, and gases from water, for the purpose of providing water with an acceptable purity for use in dialysis fluid. Purification may or may not involve sterilization. To the extent that purification is performed as an additional or optional processing step in the following disclosure, such purification may apply one or more of: a sediment filter; a carbon filter; one or more resin beds, where the resin beds may include cation resin, anion resin and mixed bed resin; ultrafiltration (UF); reverse osmosis (RO); nanofiltration; electrodeionization (EDI), or capacitive deionization (CDI).

As used herein, “ambient air” refers to air located outside of the system described herein, for example within a confined space, such as an apartment, a room, or the like. When referring to an air stream being taken from ambient air, the air stream is received from the surroundings of the system. Similarly, when referring to an air stream being emitted to ambient air, the air stream is emitted into the surroundings of the system.

As used herein, “water recovery efficiency” or “recovery efficiency” denotes the water extraction capacity of a forward osmosis (FO) process and is given by the fraction of the available water in the fluid on a feed side of an FO unit that is transported through the FO membrane to the fluid on a draw side of the FO unit. The recovery efficiency is also denoted “water extraction capacity” or simply “capacity” herein.

Like reference signs refer to like elements throughout.

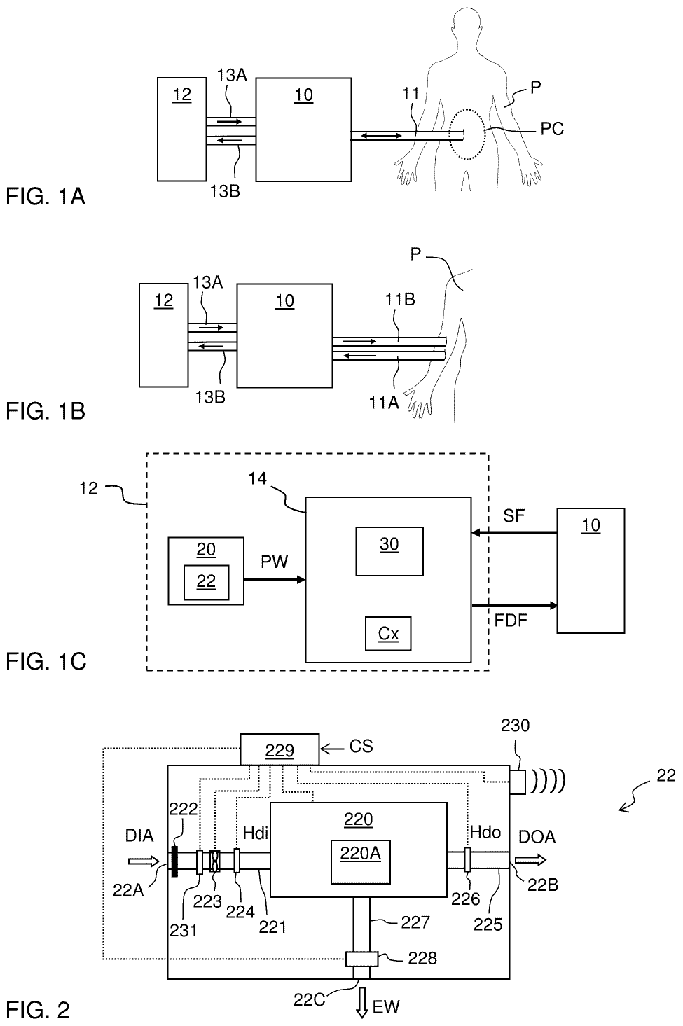

The present disclosure relates to a technique of generating dialysis fluid for a dialysis system. The technique is applicable to both peritoneal dialysis (PD) therapy and extracorporeal (EC) blood therapy. For context only, fluid generation in relation to PD therapy and EC blood therapy will be briefly discussed with reference to FIGS. 1A-1B.

FIG. 1A is a generic overview of a dialysis system for PD therapy. The dialysis system comprises a therapy system 10, which is fluidly connected to the peritoneal cavity PC of a patient P. As indicated by a double-ended arrow, the therapy system 10 is operable to convey fresh dialysis fluid into the peritoneal cavity PC and to receive spent dialysis fluid from the peritoneal cavity on a fluid path 11. The fluid path 11 may be defined by tubing that connects to an implanted catheter (not shown) in fluid communication with the peritoneal cavity PC. The therapy system 10 may be configured for any type of PD therapy. In one example, the therapy system 10 comprises one or more containers that are manually handled to perform CAPD. In another example, the therapy system 10 comprises a dialysis machine (“cycler”) that performs the dialysis therapy. The dialysis system further comprises a fluid preparation system, FPS, 12, which is configured to generate dialysis fluid for use by the therapy system 10. The dialysis fluid is supplied from the FPS 12 to the therapy system 10 on a first fluid path 13A. At least part of the spent dialysis fluid is returned to the FPS 12 on a second fluid path 13B. In embodiments described hereinbelow, the spent dialysis fluid may be used by the FPS 12 when generating the fresh dialysis fluid.

FIG. 1B is a generic overview of a dialysis system for EC blood therapy. The dialysis system comprises a therapy system 10, which is fluidly connected to the vascular system of a patient P on a fluid path. In the illustrated example, the fluid path is defined by tubing 11A for blood extraction and tubing 11B for blood return. As indicated by arrows, the therapy system 10 is operable to draw blood from the patient P through tubing 11A, process the blood, and return the processed blood to the patient through tubing 11B. The tubing 11A, 11B is connected to an access device (for example a catheter, graph or fistula, not shown) in fluid communication with the vascular system of the patient P. The therapy system 10 may be configured to process the blood by any form of EC blood therapy, such as HD, HF or HDF. In such therapy, dialysis fluid is consumed. The dialysis fluid is supplied from the FPS 12 to the therapy system 10 on the first fluid path 13A. At least part of the spent dialysis fluid is returned to the FPS 12 on the second fluid path 13B and be used by the FPS 12 when generating the fresh dialysis fluid.

FIG. 1C depicts a dialysis system in more detail, in particular the FPS 12. In accordance with some embodiments, the FPS 12 comprises a water supply unit 20, WSU, which is configured to supply process water, PW, to a fluid generation unit, FGU, 14. The WSU 20 comprises a dehumidifier unit, DHU, 22. The DHU 22 is configured to extract liquid water, EW, from incoming air. In other words, the DHU 22 is configured to harvest liquid water from moisture that is present in ambient air as vapor. PW may consist entirely of EW. However, it is conceivable that PW comprises other water in addition to EW, for example purified tap water. The WSU 20 may be configured to ensure that PW has a sufficient purity to comply with quality requirements for water to be included in dialysis fluid, for example according to ISO 23500-3. The FGU 14 is configured to mix PW, optionally after further processing, with one or more dialysis concentrates Cx to yield a final dialysis fluid, FDF, which is supplied to the therapy system 10 for use in the dialysis therapy. The FGU 14 may be configured to produce FDF in batches or on-demand. In on-demand production, the production rate of FDF is adapted or matched to the consumption rate of FDF by the therapy system 10. It is to be understood that the WSU 20 and/or the FGU 14 may comprise sterilization equipment to ensure that FDF complies with microbial requirements for dialysis fluid, for example according to aforesaid ISO 23500-3.

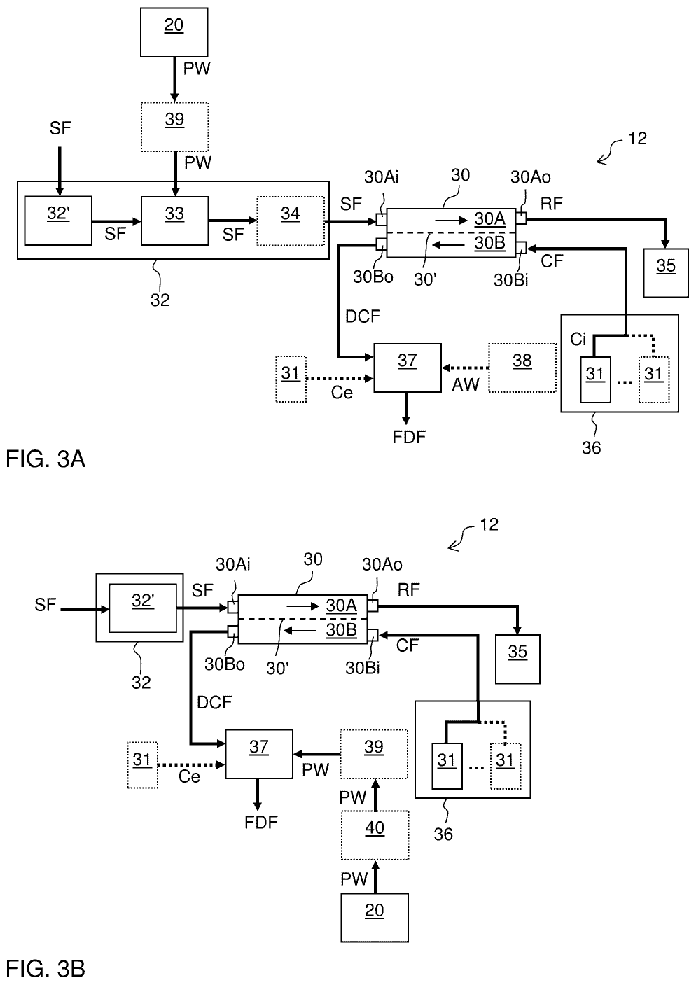

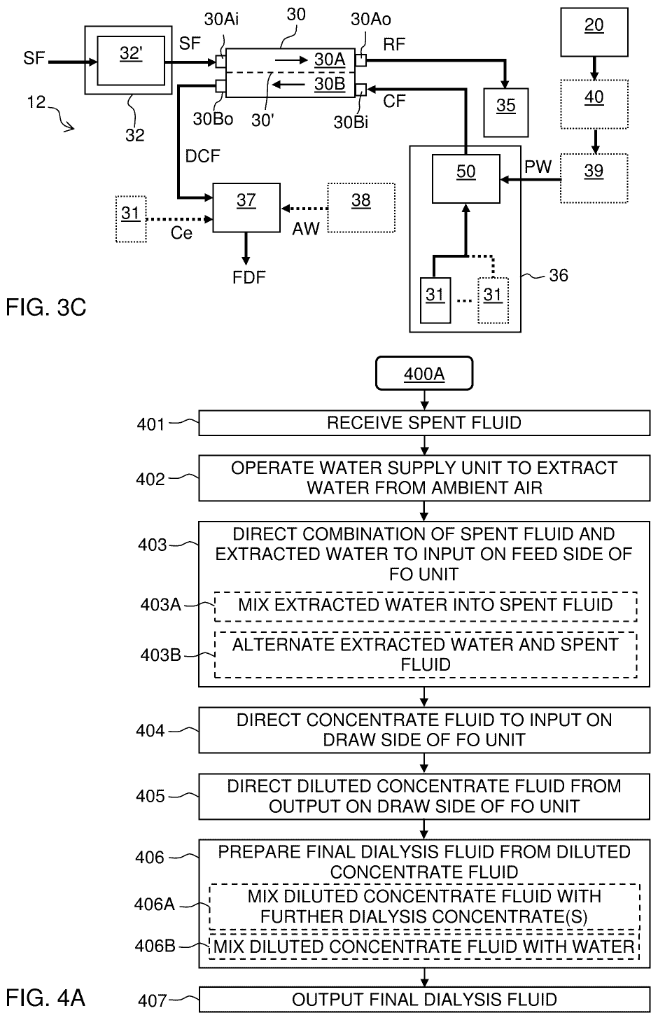

As shown in FIG. 1C, the FGU 14 is further configured to receive spent fluid, SF, from the therapy system 10. Generally, SF comprises at least spent dialysis fluid, which is dialysis fluid that contains waste, toxins and excess water that has been removed from the patient as a result of the dialysis therapy. SF may also comprise any other non-pure fluid that is produced by the therapy system 10 before, during or after dialysis therapy, for example priming fluid that may be used for purging flow paths in the therapy system 10 of air, or cleaning fluid that may be used for sanitization of flow paths in the therapy system 10. As will be described below with reference to FIGS. 3A-3C, the FGU 14 is configured to process SF by forward osmosis to extract water from SF while diluting at least one dialysis concentrate. To this end, the FGU 14 comprises at least one forward osmosis (FO) unit 30. By the combination of forward osmosis and water harvesting from ambient air, the FPS 12 is capable of producing dialysis fluid with minimum, or even no, use of tap water. If no tap water is needed, the dialysis system is “self-sustaining” with respect to water. EW extracted from ambient air will supplement the water extracted from the SF. EW may be used in production of FDF, as well in priming and/or sanitization of the therapy system 10 and/or the FPS 12. The DHU 22 and the FO unit 30 may be operated to balance the production of EW in the DHU 22 and the water extraction in the FO unit 30 to meet a total need of water. Such balanced production enables repeated generation of FDF for consecutive therapy sessions without any supply of tap water. Further, at initial start-up of the dialysis system, for example after installation, the DHU 22 may be operated to extract a sufficient amount of EW to generate FDF for an initial therapy session, whereupon the DHU 22 may be operated to provide sufficient amounts of EW to sustain repeated generation of FDF for subsequent therapy sessions. Alternatively, at least part of the FDF for the initial therapy session may be provided as a ready-made dialysis solution, for example from a pre-filled bag.

Typically, a large proportion of SF is water. This water is available for extraction in the FO unit 30. Thus, the FO unit 30 may be operated to extract a major fraction of the total water that is needed by the dialysis system. Under these circumstances, the DHU 22 only needs to supply smaller amounts of EW, which may be achievable even at adverse conditions for water harvesting, such as when the ambient air is dry and cold. At the same time, EW from the DHU 22 supplements the water extracted in the FO unit 30. Thus, compared to an installation without the DHU 22, the FO unit 30 will be operated with a smaller water extraction rate, which may extend the life of the FO unit 30 and/or reduce its need for service and maintenance.

The extraction of water in the FO unit 30 will reduce the residual volumes of SF to be handled as a result of a therapy session. For example, the FO unit 30 may reduce the volume of SF by 50-90%. The reduced need for tap water and the reduced volumes of SF have the potential of facilitating installation of the dialysis system. For example, the need to connect the dialysis system by tubing to a tap water source and/or drain may be obviated. Any small volume of tap water and/or SF may be carried by a user to and from the dialysis system without difficulty.

The mobility of the dialysis system may also be increased by the combination of DHU 22 and FO unit 30, by reducing the amount of fluid that needs to be transported together with a mobile dialysis system to render it operable. When the mobile dialysis system is self-sustaining, only the concentrate(s) used by the mobile dialysis system needs to be transported.

FIG. 2 is a block diagram of an example dehumidifier unit, DHU, 22. A water extraction unit 220 is configured to receive and process an incoming air stream DIA to change the phase state from gaseous to liquid, for at least part of the included moisture. Thereby, EW is extracted from DIA, and an outgoing air stream DOA with reduced humidity is generated. In some embodiments, the water extraction unit 220 is configured to extract EW by direct condensation of the moisture in the incoming air stream, by cooling the air below its dew point, optionally at elevated pressure. For example, the water extraction unit 220 may comprise a conventional cooling element, such as an evaporator coil, which is arranged to cool DIA, causing water to condense. In these embodiments, box 220A represents the cooling element. This type of water extraction is mainly effective when the incoming air has a high relative humidity, such as above approximately 40%. Generally, the quality of EW obtained by this technique is dependent on the quality of the incoming air.

In some embodiments, the water extraction unit 220 is instead configured to extract EW by use of a desiccant. In these embodiments, box 220A represents the desiccant. The desiccant is a hygroscopic substance which is arranged to interact with DIA. During this interaction, the desiccant absorbs and/or adsorbs water molecules that are present in DIA. The water extraction unit 220 is configured to process the desiccant 220A to release the water molecules, for example by one or more of heating, moisture vapor pressure change, or UV irradiation. The released water molecules are then collected to form EW. This type of water extraction is effective also when the incoming air has a low relative humidity, such as down to 20%, or even lower. Generally, the quality of EW obtained by this technique is dependent on the desiccant, in particular its selectivity towards water.

The DHU 22 in FIG. 2 comprises an air inlet 22A, an air outlet 22B, and a water outlet 22C. The air inlet 22A opens to an air inlet channel 221, which extends to the water extraction unit 220. An air filter 222 is arranged in the air inlet channel 221 for removal of particulate matter, such as debris, dust, etc., and possibly also volatile organic components, carbon, sub-micrometer particles, etc. A pumping device 223 is arranged downstream of the air filter 222, to generate and drive DIA through the water extraction unit 220. A humidity sensor 224 is arranged in the air inlet channel 221 to sense the inlet humidity Hdi of DIA. The air outlet 22B opens to an air outlet channel 225, which extends from the water extraction unit 220. A humidity sensor 226 is arranged in the air outlet channel 225 to sense the outlet humidity Hdo of DOA. The water outlet 22C opens to a water outlet channel 227 which extends to the water extraction unit 220. A flow controller 228 is arranged in the water outlet channel 227 to control the flow of EW from the water extraction unit 220. The flow controller 228 may for example comprise a valve and/or a pumping device. A local control unit 229 is configured to generate control signals for the pumping device 223, the flow controller 228, and the water extraction unit 220, based on sensor data from the humidity sensors 224, 226, to achieve control data CS. For example, the control data CS may include a target value for the outlet humidity Hdo and/or a target value for the flow rate of EW (“EW production rate”). The target value for the EW production rate may be set to ensure that a sufficient amount of EW is extracted over a predefined time period, for example 24 hours. The local control unit 229 may, for example, control the flow rate of DIA based on the inlet humidity Hdi to achieve the target value of the EW production rate. Alternatively or additionally, the local control unit 229 may control the flow rate of DIA based on the inlet humidity Hdi to achieve the target value of the outlet humidity Hdo. Alternatively or additionally, the local control unit 229 may control the operation of the DHU 22 to ensure that the temperature of the ambient air is within limits, for example based on a measurement signal from a temperature sensor 231 in the air inlet channel 221. The local control unit 229 may be configured to account for the size of the premises in which the DHU 22 is located, and possibly the turnover frequency of air in the premises, when determining how to control the DHU 22 to meet target values. This data represents the available volume of ambient air and may be entered by the user into the local control unit 229 via an interface device (not shown) such as a keypad, keyboard, touch screen, control buttons, etc. Alternatively, the size of the premises may be estimated by the control unit 229 based on sensor data from a room sensor 230, which may be part of the DHU 22, as shown, or a separate sensor. The room sensor 230 is configured to sense the spatial extent of the premises, by sensing spatial structures around the WSU 20. For example, the room sensor 230 may comprise one or more of a lidar sensor, a radar sensor, an imaging sensor, a range sensor, a sonar sensor, etc. If the volume of the premises is small, the control unit 229 may infer that the available volume of ambient air is small and operate the DHU 22 accordingly, and vice versa. In an alternative, the DHU 22 may at least partly obtain DIA from outside air.

The local control unit 229 may also be configured to account for the available operating time of the DHU 22, when determining how to control the DHU 22 to meet target values. For example, the WSU 20 may be scheduled to extract EW during daytime only, to avoid that the DHU 22 generates sound during nighttime.

To ensure the purity of EW, the FPS 12 (FIG. 1C) may include a dedicated purification device, which may apply any conventional water purification technique. However, a sufficient purity of EW may be attained by proper design of the DHU 22, thereby obviating the need for the dedicated purification device, at least with respect to purifying EW. If the DHU 22 is configured to extract EW from a desiccant, it is possible to achieve an inherent purification of EW by use of a desiccant that has a high selectivity towards water. The high selectively implies that the desiccant is tailored to adsorb and/or absorb water molecules rather than other molecules that may be present in DIA.

In some embodiments, the water extraction unit 220 is configured to produce EW that has conductivity of less than 10 μS/cm, and preferably less than 5 μS/cm or 1 μS/cm. As understood from the foregoing, this may be achieved by using a desiccant with a high selectivity of water. Under certain circumstances, a DHU 22 operating by direct condensation may also produce EW of sufficient purity.

In some embodiments, the desiccant is an ionic or covalent porous solid, including but not limited to metal-organic and organic porous framework materials, zeolites, organic ionic solids, inorganic ionic solids, organic molecular solids, or inorganic molecular solids, or any combination thereof. The desiccant may be used in a pure, single-phase form, as a composition of different active chemical materials, and/or in combination with performance enhancing additives modulating its properties. Performance enhancing additives may include materials with a high thermal conductivity and molar water absorptivity. The active chemical compound may be used in the form of a powders, extrudates, molded bodies, pressed pellets, pure or composite films, or sintered bodies. In some embodiments, the water capture material comprises an active chemical compound, such as a metal-organic framework (MOF). MOFs are porous materials that have repeating secondary building units (SBUs) connected to organic ligands. In some variations, the SBUs may include one or more metals or metal-containing complexes. In other variations, the organic ligands have acid and/or amine functional group(s). In certain variations, the organic ligands have carboxylic acid groups. Any MOF capable of adsorbing and desorbing water may be employed in the systems provided herein. In some embodiments, MOF-303 is used as desiccant. MOF-303has a structure of Al(OH)(HPDC), where HPDC stands for 1H-pyrazole-3,5-dicarboxylate. Other conceivable MOFs for use as desiccant include, for example, MOF-801, MOF-841 and MIL-160. A combination of MOFs may also be used as desiccant. Further examples and implementation details are found in the articles “Metal-Organic Frameworks for Water Harvesting from Air”, by Kalmutzki et al., published in Adv. Mater. 2018, 30, 1704304, and “Practical water production from desert air”, by Fathich et al., published in Sci. Adv. 2018, Vol. 8, Issue 6, which are incorporated herein by reference.

In the following, embodiments of the FPS 12 are presented with reference to FIGS. 3A-3C. The embodiments differ by how PW, which is supplied by the WSU 20, is used in the FPS 12. Since FIGS. 3A-3C are focused on the fluid flows through the FPS 12, all devices for conveying the fluids through the FPS 12, such as fluid lines, valves and pumps, have been omitted for clarity of presentation.

FIG. 3A shows an example of a first embodiment of the FPS 12, in which PW from the WSU 20 is supplied together with SF to an FO unit 30. The FO unit 30 comprises a feed side 30A and a draw side 30B separated by an FO membrane 30′. The membrane 30′ is a water-permeable membrane. The membrane 30 typically has a pore size in the nanometer (nm) range, for example from 0.5 to 5 nm or less depending on the solutes that are intended to be blocked. It separates the feed side 30A from the draw side 30B. The different sides may also be referred to as compartments. The FO unit 30 is arranged to carry different fluids on the different sides of the membrane 30. The fluids typically flow in countercurrent flows, as shown by arrows, but may alternatively flow in co-current flows. The flows may be continuous or intermittent through the FO unit 30. In some embodiments, as shown, the fluids flow a single pass, wherein the respective fluid passes through the FO unit 30 only once. Suitable FO units for FO unit 30 may be provided by Aquaporin™, AsahiKASEITM, Berghof™, CSM™, FTSH2O™, Koch Membrane Systems™, Porifera™, Toyobo™, AromaTech™, or Toray™. Water in the fluid on the feed side 30A (feed solution) is transported over the membrane 30′ to the fluid on the draw side 30B (draw solution) by the driving force created by the osmotic pressure gradient between the feed solution and the draw solution. The FO membrane 30 may be designed to be more or less exclusively selective towards water molecules, which enables the membrane 30 to separate water molecules from other molecules, as well ions and larger particles, that may be present in the feed solution. Thus, the FO membrane 30 may be configured to perform an inherent purification of water that is transported through the FO membrane 30. In some embodiments, the FO membrane 30 may separate water from other contaminants, including microbiological contamination such as bacteria, yeast, mold, fungi, virus, prions, protozoa or their toxins and by-products. Thus, the FO membrane 30 may be configured to perform an inherent sterilization of water that is transported through the FO membrane 30. The geometry of the membrane 30′ may be flat-sheet, tubular or hollow fiber.

The FO unit 30 comprises at least one input port 30Ai and at least one output port on the feed side 30A, and at least one input port 30Bi and at least one output port 30Bo on the draw side 30B. The FO unit 30 is configured to receive a concentrate fluid, CF, on the draw side 30B and to receive spent fluid, SF, on the feed side 30A to transport water from SF to CF through the membrane 30′. CF is therefore diluted to form a diluted concentrate fluid, DCF. This also means that the incoming SF will become more concentrated throughout the FO process, resulting in residual fluid, RF.

In FIG. 3A, the FPS 12 may be seen to include an SF supply arrangement (first sub-system) 32 for providing SF to the feed side 30A, a CF supply arrangement (second sub-system) 36 for providing CF to the draw side 30B, and a preparation arrangement 37 (third sub-system) for receiving DCF from the draw side 30B and processing DCF into the final dialysis fluid, FDF. In FIG. 3A, the first sub-system 32 is thus fluidly connected to the input port 30Ai, the second sub-system 36 is fluidly connected to the input port 30Bi, and the third sub-system 37 is fluidly connected to the output port 30Bo. A receptacle or drain 35 is fluidly connected to the output port 30Ao to receive the residual fluid, RF.

The first sub-system 32 is fluidly connected to receive process water, PW, from the WSU 20, optionally via a storage unit (“PW container”) 39, for example a bag or tank. The PW container 39, if present, is configured for intermediate storage of PW and serves to decouple the production rate of PW by the WSU 20 from the consumption rate of SF by the first sub-system 32. Such decoupling may facilitate control and simplify system design. The first sub-system 32 comprises an input section 32′ for receiving SF from the therapy system (cf. 10 in FIG. 1C). The input section 32′ may or may not comprise a storage unit (“SF container”) for intermediate storage of SF. A combination section 33 is arranged to receive and combine PW and SF and provide the combination to the FO unit 30, optionally via a storage unit (“SF container”) 34. The SF container(s) may be provided to decouple the infeed of spent fluid to the first sub-system 32 from the consumption rate of spent fluid in the FO unit 30. Again, such decoupling may facilitate control and simplify system design. It is understood that the spent fluid may change composition when passing the first sub-system 32. However, for the purpose of the following discussion and in FIG. 3A, spent fluid is colloquially represented as SF upstream of the FU unit 30.

The second sub-system 36 is configured to supply the above-mentioned concentrate fluid, CF. The second sub-system 36 comprises one or more containers 31 with a respective dialysis concentrate Ci, which is to be included in the final dialysis fluid, FDF. The second sub-system 36 is therefore operable to include the concentrate(s) Ci in CF. The concentrate(s) Ci may or may not be mixed with water by the second sub-system 36 to form CF. In the first embodiment, such water (if used) is not provided by the WSU 20 and is denoted auxiliary water (denoted AW). For example, a concentrate that is available to the second sub-system 36 in solid form, for example as a powder, may be mixed with water in the second sub-system 36 to form part of CF. A concentrate that is available in liquid form need not, but may, be mixed with water. Irrespective of implementation, the second sub-system 36 is configured to generate CF to include one or more liquid dialysis concentrates Ci.

Generally, as used herein, auxiliary water AW may, for example, be purified water from a pre-filled bag, tap water from a tap water source, or purified tap water supplied by a water purification unit (not shown).

The third sub-system 37 is configured to receive and process the diluted concentrate fluid, DCF, into the final dialysis fluid, FDF. As shown, the third sub-system 37 may be configured to obtain one or more concentrates Ce from one or more containers 31 (one shown), and/or obtain AW from a water source 38. If provided, AW has a quality that is acceptable for use in dialysis fluid, or is processed by the third sub-system 37 into such a quality. The processing by the third sub-system 37 may comprise one or more of mixing DCF with the dialysis concentrate(s) Ce, mixing DCF with AW, adjusting the temperature of FDF, degassing FDF, etc. Thus, in some implementations, the third sub-system 37 may not change the composition of DCF to produce FDF, but rather adjust one or more other properties.

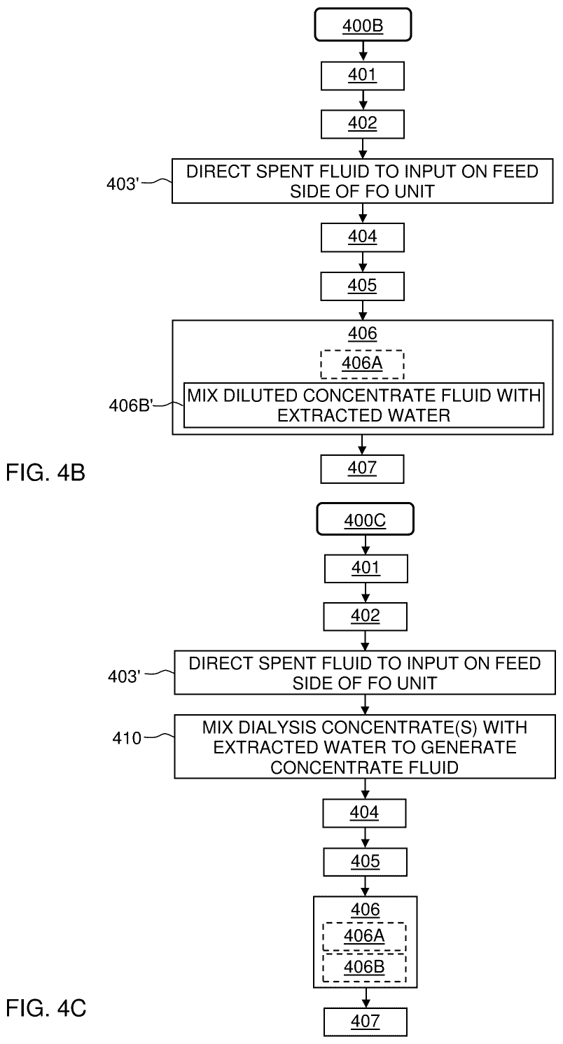

FIG. 4A is a flow chart of an example method 400A of operating the first embodiment in FIG. 3A, under the assumption that PW is only provided to the first sub-system 32. Although not shown on the drawings, it is assumed that the FPS 12 is operated by a control arrangement, which may be centralized or distributed in any form. For example, the control arrangement includes the functionality of the local control unit 229, as described with reference to FIG. 2 above.

In step 401, SF is received by the first sub-system 32, directly or indirectly, from the therapy system 10. In step 402, the WSU 20 is operated to extract water from ambient air and provide PW for receipt by the first sub-system 32. As noted, PW may be collected in a PW container 39, if present. In step 403, the first sub-system 32 is operated to generate a combination of SF and PW, in section 33, and direct the resulting combination to the input port(s) 30Ai on the feed side 30A of the FO unit 30, optionally via the SF container 34. As indicated, the combination may be generated in two different ways. In step 403A, section 33 is configured to mix PW with SF to form an SF mixture, which is provided to the FO unit 30. In step 403B, section 33 is configured to alternately provide either PW or SF to the FO unit 30. In other words, in step 403B, the first sub-system 32 is operated to relay the incoming SF to the FO unit 30 during a first time period, and to relay incoming PW to the FO unit 30 during a second time period before or after the first time period. The first and second time periods may be of any length and may be repeated any number of times during operation of the FPS 12. It is to be understood that an osmotic pressure gradient will be established across the membrane 30′ also when there is PW on the feed side 30A. Thus, water will be transferred from PW through the membrane 30′ to dilute CF on the draw side 30B.

Section 33 may be pre-configured to perform one of step 403A and step 403B. Alternatively, section 33 may be operable, by the control arrangement, to switch between step 403A and step 403B. In step 404, CF is directed from the second sub-system 36 to the input port(s) 30Bi on the draw side 30B of the FO unit 30. In step 405, DCF is directed from the output port(s) 30Bo on the FO unit 30 to the third sub-system 37. Although not shown in FIG. 4A, the method 400A also comprises a step of providing RF from the outlet port(s) 30Ao to the receptacle/drain 35.

It is understood that steps 401-405 may be performed at least partly concurrently to produce DCF, although PW may be produced by the WSU 20 and supplied to the PW container 39 in advance of the other steps.

In step 406, the third sub-system 37 is operated to produce FDF from DCF. In some embodiments, as indicated by step 406A, the third sub-system 37 is operated to mix DCF with one or more further concentrates (cf. Ce in FIG. 3A). In some embodiments, as indicated by step 406B, the third sub-system 37 is operated to mix DCF with auxiliary water (cf. AW in FIG. 3A). Steps 406A and 406B may also be combined. The operation of the third sub-system 37 will differ depending on FDF to be produced. In step 407, the third sub-system 37 is operated to output FDF, which may then be directly or indirectly supplied to the therapy system 10.

One technical advantage of supplying PW to the FPS 12 via the first sub-system 32 is that PW will be inherently subjected to the same purification and/or sterilization as SF, by the membrane 30′ in the FO unit 30. Thus, the requirement on the quality of PW, in terms of purity and/or sterility, is mitigated. This may, in turn, enable a simplified construction of the DHU 22 in the WSU 20 and/or reduce the need to install supporting equipment for purification and/or sterilization of PW.

Another technical advantage is that the provision of PW on the feed side 30A of the FO unit 30 may increase the difference in concentration between the feed and draw sides 30A, 30B and thereby increase the osmotic pressure gradient across the membrane 30. This would result in an increased rate of transport through the membrane 30′ and may, for example, enable a higher fluid flow rate on the feed side 30A and/or the draw side 30B for a given dilution of CF. Alternatively or additionally, it may enable an increased dilution of CF within the FO unit 30 in a given period of time.

FIG. 3B shows an example of a second embodiment of the FPS 12, in which PW from the WSU 20 is supplied for use in transforming DCF into FDF. The second embodiment is identical to the first embodiment in FIG. 3A with respect to the configuration of the FO unit 30, the second sub-system 36 and the drain/receptacle 35. In contrast to the first embodiment, the first sub-system 32 is configured to provide undiluted SF to the input port(s) 30Ai on the feed side 30A of the FO unit 30. In the example of FIG. 3B, the first sub-system 32 comprises an input section 32′ for receiving incoming SF from the therapy system and relaying SF to the FO unit 30. The input section 32′ may or may not comprise a storage unit (“SF container”) for intermediate storage of SF.

In further contrast to the first embodiment, the third sub-system 37 is not only fluidly connected to the FO unit 30, to receive DCF, but also fluidly connected to the WSU 20, to receive PW. PW is consumed when the third sub-system 37 produces FDF. Specifically, the third sub-system 37 is configured to include PW in FDF. In one example, PW is directly mixed into DCF, or vice versa. As shown, the third sub-system 37 may also be configured to receive one or more dialysis concentrates, represented as Ce. The dialysis concentrate(s) Ce may be mixed with PW and/or DCF. If at least one dialysis concentrate is in solid form, it is conceivable that the dialysis concentrate is mixed with PW to form a liquid concentrate, which is then mixed with DCF. It is to be understood that the third sub-system 37 may be configured to perform further processing, such as heating, degassing, etc., before outputting FDF.

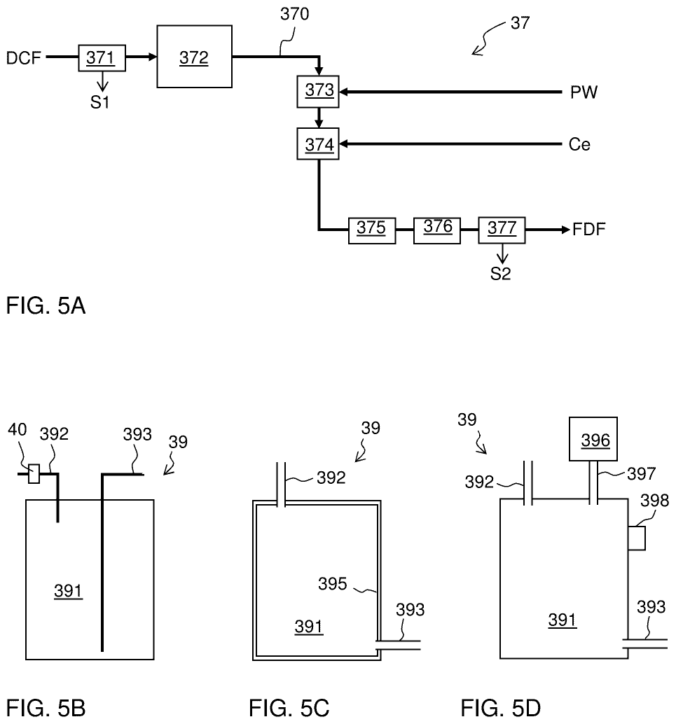

FIG. 5A shows an example configuration of the third sub-system 37. In the illustrated example, FDF is produced by mixing DCF with PW and one liquid concentrate Ce. The third sub-system 37 in FIG. 5A comprises, in sequence along a main flow path 370 through the third sub-system 37, a first conductivity sensor 371, a storage unit (“DCF container”) 372, a first mixing section 373, a second mixing section 374, a heating device 375, a mixing/degassing device 376 and a second conductivity sensor 377. The DCF container 372 is arranged for intermediate storage of incoming DCF. The provision of the DCF container 372 will serve to decouple the production rate of FDF from the generation rate of DCF by the FO unit 30, for example to enable production of DCF when there is no demand for FDF. This facilitates control of the FPS 12. The DCF container 372 may also facilitate on-demand production of FDF by the third sub-system 37. The first conductivity sensor 371 is operable to generate a conductivity signal S1, which is representative of the conductivity of DCF. DCF is pumped from the DCF container 372 along the main flow path through the first mixing section 373. The first mixing section 373 is arranged to receive PW and admix PW with DCF. The mixture of DCF and PW is transferred along the main flow path to the second mixing section 374. The second mixing section 374 is arranged to receive Ce and admix Ce with the mixture of DCF and PW, thereby producing a fluid that has the final composition of the FDF. The fluid is then conveyed through the heating device 375, which is operated to adjust the temperature of the fluid, and through the mixing device 376, which is operable to ensure homogeneity of the fluid and possibly to remove any gases released from the fluid (“degassing”). Downstream the devices 375, 376, the fluid (FDF) passes through the second conductivity sensor 377, which is operable to generate a conductivity signal S2 that represents the conductivity of the fluid. The third sub-system 37 may be operated by the above-mentioned control arrangement (not shown) to generate FDF based on the signals S1, S2. The conductivity of DCF in the container 372 is given by signal S1, and the conductivities of Ce and PW may be predefined or otherwise known, thereby allowing the control arrangement to control fluid pumps (not shown) to set the flow rates of DCF, PW and Ce so that the conductivity of FDF, as given by signal S2, meets a target value. In an alternative, the control arrangement is configured to perform feedback control of the flow rate of DCF based on S2, while PW and CE are dosed volumetrically into the main flow path 370. The procedure in FIG. 5A of first mixing the DCF with PW to generate a fluid mixture and then mixing the fluid mixture with the concentrate Ce may be advantageous since it separates the dilution of DCF from the admixing of Ce. This may facilitate process control based on conductivity signals S1, S2. Particular advantages may be achieved if DCF is prepared from concentrate(s) Ci having a higher conductivity than the respective concentrate Ce that is admixed by the third sub-system 37. For example, a dialysis fluid for PD therapy may be generated by mixing one or more concentrates comprising electrolytes and one or more concentrates comprising an osmotic agent. Generally, the electrolytes have a larger impact on conductivity than the osmotic agent and may therefore be at least partly included in DCF. A dialysis fluid for EC blood therapy may be generated by mixing one or more concentrates comprising electrolytes and one or more concentrates comprising a buffer. Generally, the electrolytes have a larger impact on conductivity than the buffer and may therefore be at least partly included in DCF.

The skilled person understands that the conductivity sensors 371, 377 in FIG. 5A may be replaced by any sensor capable of measuring an equivalent property. Thus, within the context of the present disclosure, no distinction is made between a concentration sensor, a conductivity sensor and a resistivity sensor.

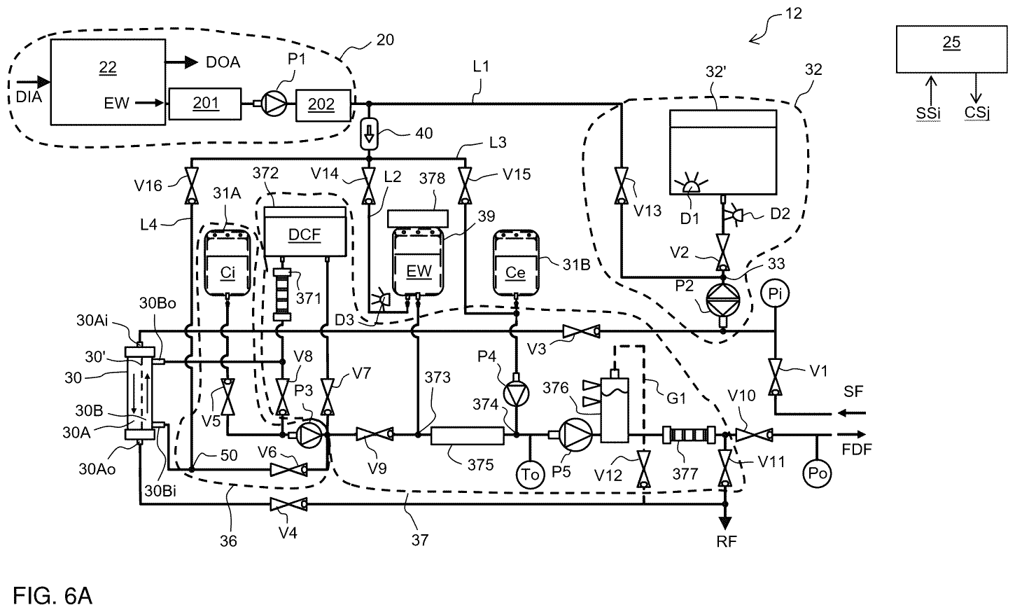

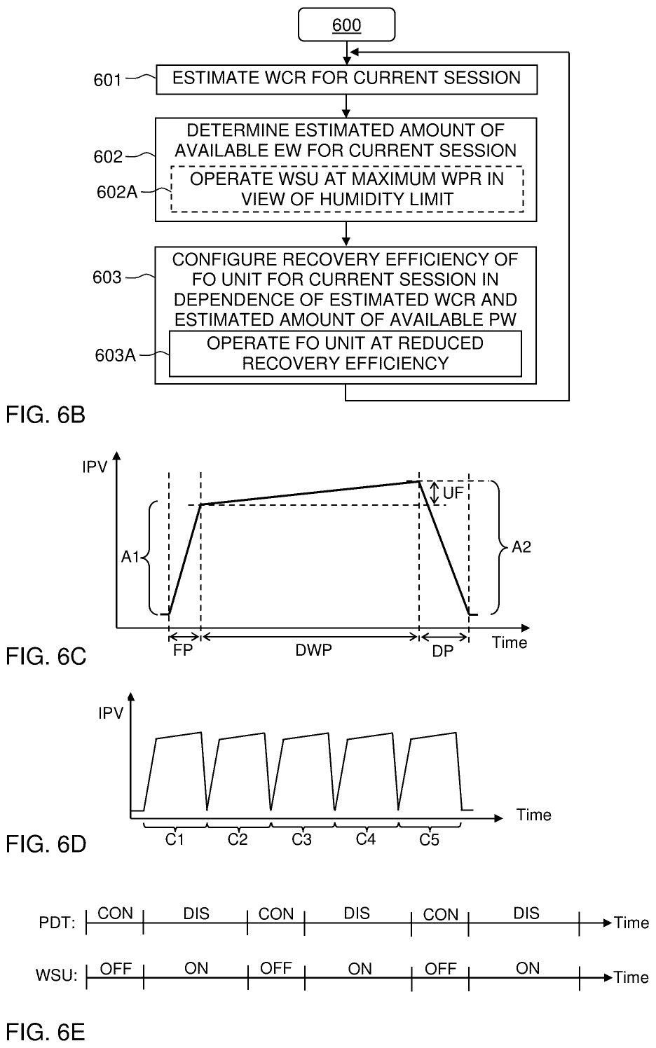

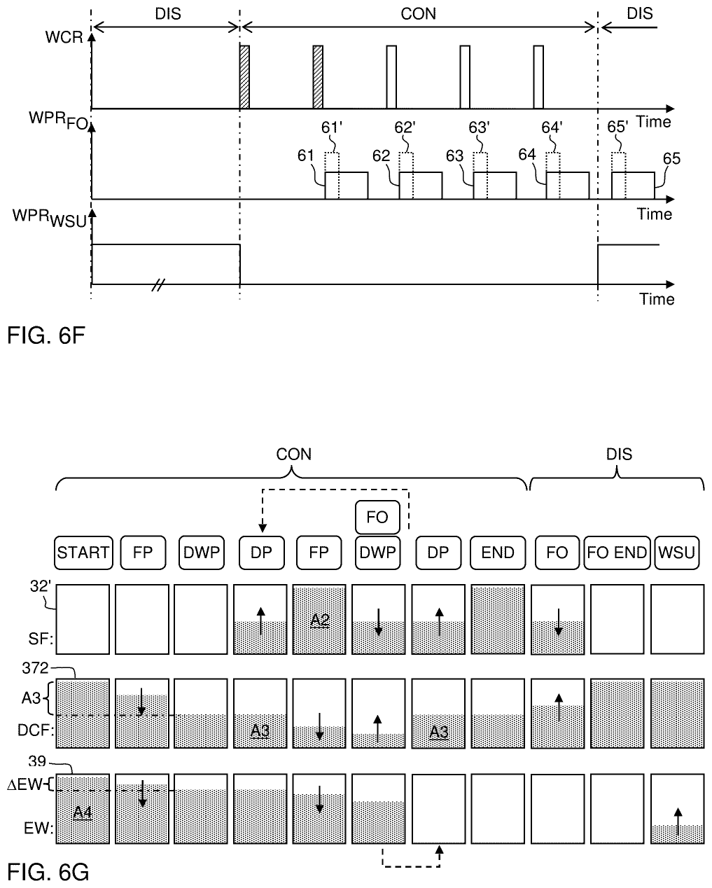

The first and second mixing sections 373, 374 may but need not be tailored to promote mixing. For example, a mixing section may comprise a recirculation system, a mixing tank, an agitation device, etc. It is also conceivable that the mixing section is merely a juncture and a downstream section of a fluid channel, where two fluids meet to be at least partly mixed. Such junctures are included in the detailed example of FIG. 6A (below). The configuration of respective mixing section 373, 374 may differ depending on the properties of the fluids to be mixed and may also differ depending on whether FDF is produced batch-wise or on-demand. In any event, it is to be understood that FIG. 5A is given as a non-limiting example. The heating and/or degassing may be omitted in some implementations. For example, in batch production, the temperature adjustment of the FDF may be omitted. The configuration in FIG. 5A may be adapted to enable admixing of more than one dialysis concentrate, or no dialysis concentrate. It may also be noted that the configuration in FIG. 5A is applicable also to the first embodiment in FIG. 3A and the third embodiment in FIG. 3C (below), albeit with PW replaced by AW.

FIG. 4B is a flow chart of an example method 400B of operating the second embodiment in FIG. 3B, under the assumption that PW is only provided to the third sub-system 37. It is assumed that the FPS 12 is operated by the above-mentioned control arrangement. Many steps are identical to the steps of method 400A (FIG. 4A) and will not be reiterated. One difference over method 400A is that step 403 is replaced by step 403′, in which the first sub-system 32 is operated to direct SF to the input port(s) 30Ai on the feed side 30A of the FO unit 30 without any preceding dilution. Another difference is that optional step 406B is replaced by mandatory step 406B′, in which the third sub-system 37 is operated to mix DCF with PW in the process (step 406) of preparing FDF from DCF.

One technical advantage of supplying PW to the FPS 12 via the third sub-system 37 is to facilitate system design and control. Basically, any conventional arrangement for mixing water and one or more concentrates, for on-demand production or batch-wise production of FDF, may be implemented in the third sub-system 37.

Reverting to FIG. 3B, a PW container 39 may be included for intermediate storage of PW before it is provided to the third sub-system 37. Like in the first embodiment, such a PW container 39 serves to decouple the production of PW from the consumption of PW. Depending on the quality of PW produced by the WSU 20, the FPS 12 may also comprise a sterilization unit 40 for sterilizing PW before it is supplied to the third sub-system 37. As shown, the sterilization unit 40 may be arranged upstream of the PW container 39, if present, to reduce the need for cleaning/sterilization of the PW container 39. Alternatively or additionally, the sterilization unit 40 may be included in the third sub-system 37 to sterilize PW, to sterilize a fluid mixture that includes PW, or to sterilize FDF. It may be noted that such as sterilization unit 40 may not be needed in the first embodiment (FIG. 3A) due to the sterilizing effect of the membrane 30′ in the FO unit 30.

FIG. 3C shows an example of a third embodiment of the FPS 12, in which PW from the WSU 20 is supplied for use in generating CF. The third embodiment is identical to the first embodiment in FIG. 3A with respect to the configuration of the FO unit 30, the third sub-system 37 and the drain/receptacle 35. In contrast to the first embodiment, the first sub-system 32 is configured to provide undiluted SF to the input port(s) 30Ai on the feed side 30A of the FO unit 30. For example, the second sub-system 32 may be configured as described for the second embodiment.

In further contrast to the first embodiment, the second sub-system 36 is fluidly connected to the WSU 20, to receive PW, which is consumed when the second sub-system 36 produces CF. Specifically, the second sub-system 36 is configured to include PW in CF. In the illustrated example, the second sub-system 36 comprises a mixing section 50, in which PW is mixed with the concentrate(s) Ci for dilution. Like in the first and second embodiments, a PW container 39 may be included for intermediate storage of PW before it is provided to the second sub-system 36. Similar to the second embodiment, the FPS 12 may also comprise a sterilization unit 40 for sterilizing PW before it is supplied to the second sub-system 36. Alternatively or additionally, the sterilization unit 40 may be included in the second sub-system 36 to sterilize PW or CF.

FIG. 4C is a flow chart of an example method 400C of operating the third embodiment in FIG. 3C, under the assumption that PW is only provided to the second sub-system 36. It is assumed that the FPS 12 is operated by the above-mentioned control arrangement. Many steps are identical to the steps of method 400A (FIG. 4A) and will not be reiterated. One difference over method 400A is that step 403 is replaced by step 403′, in which SF is directed to the input port(s) 30Ai on the feed side 30A of the FO unit 30 without any preceding dilution. Step 403′ may thus be identical to step 403′ in the method 400B (FIG. 4B). Another difference is that method 400C comprises an additional mandatory step 410, in which the second sub-system 36 is operated to mix the concentrate(s) Ci with PW to generate CF.

One technical advantage of supplying PW to the FPS 12 via the second sub-system 36 is that it possible to adjust the concentration of CF, for example to affect the water transport in the FO unit 30 and/or optimize the performance of the FPS 12 as a whole.

One or more of the first to third embodiments may be combined, so that PW is provided to more than one of the first, second and third sub-systems 32, 36, 37. For example, the auxiliary water (AW) as used by the second or third sub-systems 36, 37 in the examples of FIGS. 3A-3C may be at least partly replaced by PW.

As described, the FPS 12 may include a PW container 39 for intermediate storage of PW. This container 39 may be configured to prevent or mitigate microbiological growth. Some non-limiting examples are shown in FIGS. 5B-5D.

In FIG. 5B, the PW container 39 is configured as a disposable unit, which is removably installed in the FPS 12 and is discarded a predefined time after installation or after a predefined number of therapy sessions by the therapy system 10. In the illustrated example, the disposable unit 39 defines an internal compartment 391 for FW and comprises an FW inlet line 392, which extends into the compartment 391, and an FW outlet line 393, which extends from a bottom portion of the compartment 391. In the illustrated example, a sterilization unit 40, for example a sterile filter, is arranged on the inlet line 392. Alternatively or additionally, a sterilization unit 40 may be arranged on the outlet line 393.

In FIG. 5C, the PW container 39 is a permanent component in the FPS 12. The PW container 39 defines an internal compartment 391 for PW and comprises inlet and outlet lines 392, 393 at the top and bottom, respectively, of the compartment 391. To counteract microbiological growth, the walls of the compartment 391 are lined with an antimicrobial material 395, for example a plastic material.

In FIG. 5D, the PW container 39 is a permanent component in the FPS 12. The PW container 39 is associated with permanent sterilization equipment, which is operable to sterilize the internal compartment 391 and/or PW therein. In one example, the permanent sterilization equipment comprises a heat sterilization device 396, which is connected by a fluid passage 397 to supply heated fluid to the internal compartment 391. The heated fluid may be generated from PW or AW. In another example, the permanent sterilization equipment comprises a radiation source 398, for example a UV source, which is arranged to irradiate the compartment 391 and/or PW by sterilizing radiation to counteract microbiological growth.

The examples in FIGS. 5B-5D are also applicable to other storage units in the FPS 12, for example the SF container 34 (FIG. 3A) and the DCF container 372 (FIG. 5A).

FIG. 6A shows a more detailed example of how the first to third embodiments may be implemented in an FPS 12. The operation of the FPS 12 is controlled by a control arrangement 25, which is configured to receive sensor signals SSi from sensors in the FPS 12 and generate control signals CSⱼ for operable components in the FPS 12, for example valves (enumerated with initial V) and pumps (enumerated with initial P). In FIG. 6A, the WSU 20 comprises a DHU 22, which is configured to extract liquid water EW from an incoming air stream, DIA. The WSU 20 is configured to supply only EW. Thus, in the following, EW is equivalent to PW and any reference to EW may be replaced by PW. The WSU 20 further comprises a sensor arrangement 201 for measuring one or more properties of EW. The control arrangement 25 may be configured to shut-down the FPS 12 and generate an alert if EW has a non-acceptable property. In some embodiments, the control arrangement 25 may be configured to selectively configure the FPS 12 to operate according to one or more of the first to third embodiments based on the one or more properties measured by the sensor arrangement 201. For example, if the sensor arrangement 201 indicates low purity of EW, the control arrangement 25 may switch the FPS 12 to operate in the first embodiment. A pump P1 is arranged to supply EW at a flow rate set by the control arrangement 25. A flow meter 202 may be included to measure the flow rate of EW from the WSU 20.

In the illustrated example, the WSU 20 is fluidly connected on line L1 to the first sub-system 32, on line L4 to the second sub-system 36, and on lines L2, L3 to the third sub-system 37. Approximate boundaries of the sub-systems 32, 36, 37 are indicated by dashed lines.

Before describing the use of EW in the respective sub-system, the overall structure and operation of the FPS 12 will be described. The first sub-system 32 comprises an SF container 32′, which is arranged to hold SF. In a filling stage, incoming SF is admitted into the container 32′ as valves V1, V2 are open and valves V3, V13 are closed, by the bi-directional pump P2 being operated to pump SF into the SF container 32′. The incoming SF may originate from a therapy system (10 in FIGS. 1A-1C). In an FO operation stage, valve V1 is closed and valves V2, V3, V4 are open, and the pumping direction of the pump P2 is reversed to pump SF from the SF container 32′ into a supply line 60 that extends to the feed side of the FO unit 30. Valve V13 may or may not be closed. SF is thereby pumped through the feed side of the FO unit 30, and RF is emitted into a drain line 61 at the downstream end of the feed side. By action of pump P2, RF flows through the drain line 61 via valve V4 for ejection from the FPS 12, for example into the drain/receptacle 25 shown in FIGS. 3A-3C. The second sub-system 36 comprises a replaceable container 31A, which holds a concentrate Ci. In the FO operation stage, valves V5, V6 are open and valves V7, V8, V9 are closed, and a pump P3 in the second sub-system 36 is operated to pump Ci from container 31A through valves V5 and V6 into the draw side of the FO unit 30. Assuming that no EW is admitted into the second sub-system 36, the concentrate fluid CF is equal to Ci. As a result of the water extraction by the FO unit 30, DCF is formed on the draw side and flows, by action of pump P3, into a DCF container 372 in the third sub-system 37. A first conductivity sensor 371 is arranged to measure the conductivity of the DCF (cf. FIG. 5A). In an optional mixing stage, valves V7, V8 are open and valves V5, V6 are closed, and pump P3 is operated to recirculate DCF through the container 372. The mixing stage may be terminated after a predefined time or when the conductivity measured by sensor 371 is stabilized. In a preparation stage, valves V8, V9, V10 arc open and valves V5, V6, V7, V11 are closed, and pump P3 is operated to pump DCF from container 372 into a mixing line 62 that extends to an outlet for FDF. The mixing line 62 comprises a heating device 375, which is operable to adjust the temperature of FDF, given by a temperature sensor To, to a target value. A conventional mixing device 376 is operable to ensure homogeneity of the FDF. The mixing device 376 may also enable degassing, by collecting any gases that are released from the FDF in a mixing vessel and intermittently opening valve V12 to emit the collected gases, via gas line G1, into the drain line 61. Downstream of valve V9 in the mixing line 62, EW is admitted at a first mixing section 373, and a further concentrate Ce is admitted at a second mixing section 374. In the illustrated example, pump P5 is operated to achieve a desired flow rate of FDF, and pump P4 is operated in relation to the pump P3 to achieve a target relation between the amounts of DCF and Ce. Further, pumps P3, P4, P5 arc jointly operated to draw water into the mixing line 62 at the first mixing section 373 to achieve a target conductivity at a second conductivity sensor 377 at the downstream end of the mixing line 62. Should the measured conductivity at the sensor 377 deviate from the target conductivity, valve V10 is closed and valve V11 is opened to direct FDF to drain, until the FPS 12 is operated to produce FDF with the target conductivity. In the illustrated example, pressure sensors Pi, Po are arranged to measure fluid pressure at the inlet of SF and outlet of FDF, respectively. The fluid pressures may be monitored for compliance with pressure limits.

In the example of FIG. 6A, the WSU 20 is fluidly connected to the first sub-system 32 by fluid line L1. The first sub-system 32 is operable to dilute SF by EW in accordance with the first embodiment (cf. FIG. 3A). In one implementation, SF and EW are mixed in the downstream supply line 60. For example, the control arrangement 25 may open valves V2, V13, operate pump PI to pump EW into line L1, and operate pump P2 to pump SF from container 32′ and EW from line L1. The relation between EW and SF is given by the pump speeds of pumps P1, P2. In another implementation, SF and EW are mixed in the container 32′. For example, the control arrangement 25 may open valves V2, V13, operate pump P1, and stop pump P2 (to thereby block fluid flow through P2), causing EW to flow into the container 32′. The first sub-system 32 is also operable to generate alternating flows of SF and EW to the FO unit 30, by operating pump P2 and selectively opening either valve V2 or valve V13.

The WSU 20 is fluidly connected to the third sub-system 37 by fluid line L2 and fluid line L3. Depending on the quality of EW, the FPS 12 may include a sterilizing filter 40 to ensure that EW received by the third sub-system 32 is acceptable for inclusion in dialysis fluid. Line L2 extends to an EW container 39, which is fluidly connected to the first mixing section 373 on the mixing line 62, so as to enable EW to be mixed with DCF pumped from the container 372 into the mixing line 62 in the preparation stage (cf. the second embodiment in FIG. 3B). The control arrangement 25 is operable to refill the container 39 by intermittently opening valve V14 and operating pump Pl to pump EW through line L2 into the container 39. The control arrangement 25 may monitor the amount of EW in the container 39 based on an output signal from a scale 378, which is arranged to measure the weight of the container 39. Alternatively, the scale 378 may be replaced by a conventional fluid level sensor. In a further alternative, the control arrangement 25 may monitor the amount of EW in the container 39 by calculating the consumption of EW based on the pumping speeds of pumps P3, P4, P5 in the above-mentioned preparation stage. Line L3 is fluidly connected to a fluid line that extends between a container 31B, which contains Ce, and the second mixing section 374. The control arrangement 25 is operable to dilute the concentrate Ce by opening valve V15 (and closing valves V13, V14, V16) and operating pump PI to pump EW into line L3. The relation between EW and Ce is given by the pump speeds of pumps P1, P4. In an alternative, not shown, line L3 extends to the container 31B and mixing of Ce and EW is made within the container 31B.

The WSU 20 is fluidly connected to the second sub-system 36 by fluid line L4. Again, the sterilizing filter 40 may be provided to ensure that EW meets quality requirements. The control arrangement 25 is operable to open valve V16 (and close valves V13, V14, V15) and operate pump P1 to pump EW through line L4 to the mixing section 50, at which EW is admixed with the concentrate Ci pumped from the Ci container 31 by pump P3 in the FO operation stage. Thereby, the concentrate fluid CF is formed by the resulting mixture of Ci and EW (cf. the third embodiment in FIG. 3C).

In the illustrated example, the FPS 12 comprises additional sterilization units DI, D2, D3 to mitigate microbiological growth. The sterilization units DI-D3 may be radiation sources (cf. 398 in FIG. 5D). As shown, DI may be arranged to sterilize the SF container 32′, D2 may be arranged to sterilize part of the outlet tubing that extends from the SF container 32′ to the supply line 60, and D3 may be arranged to sterilize part of line L2 and/or the EW container 39.

FIG. 6A is provided to show several examples of how EW may be provided and used in an FPS 12. It is to be understood that the FPS 12 may be reconfigured to include any subset of lines L1-L4. If line L2 is omitted, the container 39 may instead be connected to a source of the above-mentioned auxiliary water, AW.

In some embodiments, the control arrangement 25 is configured to jointly operate the WSU 20 and the FO unit 30 to achieve a target production rate of FDF. In a typical example, the target production rate of FDF is adapted to a consumption rate of FDF by a downstream therapy system, which is arranged to receive the FDF from the FPS 12 and consume the FDF in a treatment session (cf. FIGS. 1A-1C). The consumption rate of FDF may be given by a prescription for the treatment session and may, depending on dialysis therapy, be a fixed value or a time-varying value.

FIG. 6B is an example method 600 performed by the control arrangement 25 to configure the FPS 12, for example shown in FIG. 6A, to produce FDF for an upcoming treatment session of dialysis therapy. In the following, the upcoming treatment session is denoted “current session” and the latest treatment session is denoted “preceding session”.

In step 601, the water consumption rate (WCR) for the current session is estimated. The WCR designates the amount of water that is consumed at different time points during the current session and may be estimated based on the above-mentioned target production rate of FDF.

In step 602, an estimated amount of available EW is determined for the current session. The estimated amount includes the EW that is produced by the WSU 20 between the preceding session and the current session. This corresponds to estimating the starting amount of EW in the EW container 39 at the start of the current session. The starting amount may be determined by measurement and/or prediction. For example, the content of the EW container 39 may be measured by the scale 378. If the WSU 20 is active during the current session, step 602 may also comprise estimating the additional EW that is produced by the WSU 20 during the current session. The additional EW, and optionally the starting amount, may be determined by use of a calculation model for the WSU 20. The calculation model may be based on analytical functions or be a machine learning-based model. The calculation model is configured to estimate the EW production rate. The EW production rate depends on the settings of the WSU 20, which are known, and the humidity of the ambient air over time, which may be predicted. The prediction of the humidity may account for the available volume of ambient air in the premises where the WSU 20 is located. As described above, the available volume of ambient air may be given by input data entered by the user and/or provided by a room sensor 230 (FIG. 2). The control arrangement 25 may also store historic data on the EW production rate over time, for example at different times (hours, days, weeks, months, seasons, etc.) or for different humidity levels in the premises. The control arrangement 25 may use the historical data to improve the estimation of the amount of available EW.

In some embodiments, as indicated by step 602A, the WSU 20 is operated to achieve a maximum water production rate (WPR) in view of a lower humidity limit of the ambient air. The WPR is equivalent to the above-mentioned EW production rate. In other words, the WSU 12 operated to maximize the extraction of EW from the ambient air while maintaining the humidity of the ambient air above the lower humidity limit. For example, the humidity of the ambient air may be given by the inlet humidity Hdi or the outlet humidity Hdo of the DHU 22 (FIG. 2) or by a humidity value measured by a separate humidity sensor in the premises of DHU 22. The lower humidity limit may, for example be 30% or 40% RH. The lower humidity level may be predefined or input by the user. The control arrangement 25 may be configured to repeatedly monitor the humidity of the ambient air and stop water production by the WSU 20 when the humidity falls below the lower humidity limit, or when the EW container 39 is full.

The WSU 20 may also be operated in step 602A to ensure that the temperature of the ambient air is acceptable. During operation of the DHU 22 (FIG. 2), the DOA may be heated by the water extraction process and thereby cause the temperature of the surroundings to increase. Temperature limit(s) may be predefined or input by the user. For example, an upper temperature limit for ambient air may be set in the range of about 20° C.-30° C. The temperature of the ambient air may be measured by the temperature sensor 231 in the air inlet channel 221 of the DHU 22 (FIG. 2), or by a separate temperature sensor. The control arrangement 25 may be configured to repeatedly monitor the temperature of the ambient air and stop or modify the water production by the WSU 20 if the temperature exceeds the upper temperature limit.

In step 603, the FO unit 30 is configured for operation during the current session in dependence of the estimated WCR from step 601 and the estimated amount of available EW from step 602. Thus, by step 603, the operation of the FO unit 30 is actively adjusted in view of the amount of EW that is expected to be available during the current session. In some embodiments, the FO unit is operated at reduced water recovery efficiency. This means that FO unit 30 is operated to produce, for a given amount of SF, an amount of water (WPA) that is less than the maximum amount for the FO unit 30 (WPAmax). The maximum recovery efficiency, resulting in WPAmax, is achieved by optimizing the process parameters of the FO process for a given FO unit and for given fluids on the feed and draw sides. Such process parameters include the fluid flow rate on the feed side and the draw side, respectively, and the transmembrane pressure (TMP). The reduced recovery efficiency in step 603A results in an increased reject flow rate, i.c., an increased amount of residual fluid, RF. The reduced recovery efficiency is beneficial for several reasons. For example, the risk of fouling and scaling is reduced due to less up-concentration and shorter residence time of the spent fluid, SF, in the FO unit 30. The selectivity is also improved by the shorter residence time.

The method 600 in FIG. 6B is performed in advance of the current session. However, as indicated in FIG. 6B, steps 601-603 may also be repeated during the current session, where step 601 involves estimating an updated WCR for the remainder of the current session, step 602 involves determining the estimated amount of available EW for the remainder of the current session and step 603 involves adjusting the recovery efficiency of the FO unit 30 accordingly. In an alternative, step 601 is not repeated.

The method 600 is applicable to any of the first to third embodiments shown in FIGS. 3A-3C and implemented by the FPS 12 in FIG. 6A. The method 600 is also applicable to either PD therapy or EC blood therapy. The method 600 will be further explained below in relation to PD therapy with reference to FIGS. 6C-6G.

FIG. 6C schematically represents part of a PD session in terms of intraperitoneal volume (IPV) as a function of time. IPV designates the amount of fluid that resides in the PC. In a PD session, the therapy system 10 is fluidly connected to the patient P, by the fluid line 11 being connected to an access device (FIG. 1A), whereupon the therapy system 10 is operated to perform one or more fluid exchange cycles (“cycles”). After completion of the cycles, the patent P is fluidly disconnected from the therapy system 10. FIG. 6C shows a fluid exchange cycle. The cycle Cl consists of a fill phase (FP), a dwell phase (DWP) and a drain phase (DP), performed in sequence. In FP, a first amount (A1) of FDF is infused into the PC. In DWP, the FDF is left to reside in the PC. In DP, a second amount (A2) of spent fluid, SF, is withdrawn from the PC. As indicated in FIG. 6C, the spent fluid from PD therapy will typically not only contain the spent dialysis fluid but also excess water extracted from the body of the patient, also known as ultrafiltrate (UF). Thus, A2 is typically larger than A1.

FIG. 6D illustrates IPV as a function of time during a PD session. The PD session comprises a series of consecutive fluid exchange cycles, here five cycles C1-C5. It may be noted that FIG. 6D represents a PD session that is terminated by a drain phase, and the patient is “dry” between treatment sessions. The next PD session starts by a fill phase. In an alternative, not shown, the PD session is terminated by a fill phase, the patient is “wet” between sessions, and the next PD session starts by a drain phase. The examples below presume that the patient is “dry” between sessions, but the conclusions are equally applicable to PD sessions that result in a “wet” patient between sessions.

As used herein, a “treatment session” (“session”) corresponds to the time period when the patient is fluidly connected to the therapy system 10, designated by CON in the figures. Consequently, the patient is fluidly disconnected from the therapy system 10 between sessions, this time period being designated by DIS in the figures. FIG. 6E, shows an example timeline of PD therapy, PDT, and a corresponding operational state of the WSU 20. The PD therapy comprises an alternating sequence of CON and DIS. For example, the combined duration a CON and a DIS may correspond to one day (24 hours). In FIG. 6E, it is presumed that CON is performed during nighttime, while the patient sleeps (nocturnal PD therapy). To minimize disturbances for the patient, the WSU 20 is deactivated (OFF) during CON, and activated (ON) only during DIS. FIG. 6E is merely an example. The WSU 20 may alternatively be activated during nighttime as well. In another alternative, the WSU 20 is activated during daytime, irrespective of when sessions are performed. Thus, ON may at least partly coincide with CON. In a non-limiting example, CON has a duration of 7-9 hours.

FIG. 6F shows an example of the water consumption rate (WCR), the water production rate by FO (WPR_{FO}) and the water production rate by the WSU (WPR_{WSU}) during a current PD session (CON) with five consecutive cycles and “dry” patient between sessions. In the illustrated example, the WSU 20 is only active between treatment sessions (during DIS). Further, the WSU 20 is, for simplicity only, assumed to be operated in steady state so that the WSU water production rate (WPR_{WSU}) is constant over time. As will be understood from the following, WPR_{FO} and WPR_{WSU} are adapted to enable WCR. As understood from the above, WCR is the water consumption rate that is given by the consumption rate of FDF during the PD session. In FIG. 6F, the WCR is represented as five bars, each corresponding to a fill phase. The FO water production rate (WPR_{FO}) is represented by five consecutive bars 61-65, which occur subsequent to a respective drain phase. Thus, each bar 61-65 corresponds to generation of DCF from SF obtained by a drain phase. It is to be noted that the first DCF production during the session (bar 61) is performed after the second fill phase, since this is the first time during CON that SF is available. Thus, the required water for the two initial fill phases (hatched bars in FIG. 6F) typically needs to be generated by use of EW from the WSU 20 and/or extracted water by FO processing during a preceding session. The extracted water may originate from FO processing of SF from the last two drain phases in the preceding session, corresponding to bars 64, 65 in FIG. 6F. In FIG. 6F, it is assumed that the SF obtained in a drain phase is processed into DCF after the subsequent fill phase. In a variant, the SF is processed into DCF between the drain phase and the subsequent fill phase. However, this may undesirably prolong the PD session or put undesirably high demands on the FO processing.

If the patient is instead “wet” between sessions, the last bar 65 in FIG. 6F is instead located before the first bar 61 and corresponds to FO processing of SF from the initial drain. In such an implementation, only water for the first fill phase needs to be generated by use of EW from the WSU 20 and/or extracted water by FO processing during the preceding session.

A first observation based on FIG. 6F is that the joint operation of the WSU 20 and the FO unit 30, by steps 602-603 in FIG. 6B, should be performed to enable the first fill phase(s) of the current session. In the example of the second embodiment in the context of FIG. 6A, the FDF for the first fill phase(s) may be generated by use of stored EW in the EW container 39 and/or stored DCF in the DCF container 372.

As noted above, the FO unit 30 has a maximum recovery efficiency. The bars 61-65 in FIG. 6F are given for operation of the FU unit 30 at maximum recovery efficiency (“maximum water extraction capacity”). By step 603 in FIG. 6B, based on the estimated amount of available EW from the WSU 20, the FO unit 30 may instead be configured to operate at a fraction (a) of the maximum recovery efficiency, for example resulting in the dotted bars 61′-65′. It is currently believed that the life of the FO unit 30 is significantly extended when α=0.95, and is further extended with decreasing α. In some embodiments, α≤0.9 or α≤0.8. It is to be understood that a may vary during a session, depending on the estimated availability of EW.

A second observation based on FIG. 6F is thus that it is feasible to use EW from the WSU 20 to reduce the recovery efficiency of the FO unit 30.

The FO unit 30 may, when operating at maximum recovery efficiency, produce about 90%-100% of the total amount of water that is needed to produce FDF from concentrate(s). One reason for this ability is the presence of ultrafiltrate (UF in FIG. 6B) in the spent fluid. The UF is also available for extraction from SF in the FO process. The EW produced by the WSU 20, by extraction from ambient air, makes it possible to operate the FO process to produce less than about 90%, for example less than about 85%, about 80%, about 75% or about 70%, of the water needed to perform the dialysis treatment. Hence, the FO process may be operated well away from its maximum capacity.

FIG. 6G graphically depicts an example of the fill status of the SF container 32′, the DCF container 372 and the EW container 39 in the FPS 12 of FIG. 6A during a session (CON) and between sessions (DIS). The corresponding operating stages of the system are indicated at the top of FIG. 6G. In the example of FIG. 6G, it is assumed that the FPS 12 is operated in accordance with the second embodiment and thus that EW is admixed with DCF in the third sub-system 37 to generate the FDF. It is also assumed that the patient is “dry” between sessions. At start of the session, the SF container 32′ is empty, and the DCF and EW containers 372, 39 are full. The amount in the EW container 39 at the start of the session is designated by A4. In the first fill phase (FP), an amount (A3) of DCF is drawn from the DCF container 372 and mixed with a supplementary amount (AEW) of EW from the EW container 39, and optionally with further concentrate(s) Ce, to produce FDF. In the illustrated example, about half of the DCF in the DCF container 372 is consumed in FP. In the following dwell phase (DWP), the fill statuses are unchanged. In the following drain phase (DP), SF from the peritoneal cavity is pumped into the SF container 32′ by the therapy system. At the end of the DP, the SF container 32′ is full. The fill statuses of the DCF and EW containers 372, 39 are unchanged during the DP. In the next fill phase (FP), another amount (A3) of DCF is drawn from the DCF container 372 and mixed with AEW from the EW container 39. Thus, in the first two fill phases, the required amount of FDF is produced from stored DCF and stored EW (cf. hatched bars in FIG. 6F). In the next dwell phase (DWP), the FO unit 30 is operated to process SF into DCF. Thus, SF is pumped from the SF container 32′ through the feed side 30A of the FO unit 30 to produce DCF on the draw side 30B of the FO unit 30. The resulting amount (A3) of DCF is pumped into the DCF container 372. As indicated by a dashed arrow at the top of FIG. 6G, the FPS 12 may then be operated to perform one or more further repetitions of DP, FP and DWP while consuming EW from the EW container 39. As understood, for each repetition, the fluid level in the DCF container 372 is restored by the FO processing during DWP. In the illustrated example, the amount (A4) of EW in the EW container 39 is sufficient for three additional repetitions of DP, FP, DWP, resulting in a total of five cycles (cf. FIG. 6F). After the last dwell phase (DWP), as indicated by a dashed arrow at the bottom of FIG. 6G, a final drain phase (DF) is performed, in which SF is pumped into the SF container 32′. At the end of the session, the SF container 32′ is full, the DCF container 372 is half full, and the EW container 39 is empty. In the time period (DIS) between sessions, the FO unit 30 is operated to process the SF from the SF container 32′ into DCF. At the end of the FO processing, the SF container 32′ is empty, and the DCF container 372 is full and thus ready for a new session. In DIS, the WSU 20 is also activated to generate EW from ambient air and accumulate EW in the EW container 39. The WSU 20 is operated during DIS so that the EW container 39 again is full (A4) at the start of the next session.

The graphics in FIG. 6G is merely an example. In practice, the system is operated with margins so that the EW container 39 and/or the DSF container 372 never are completely emptied. Further, any of the containers need not be full, but may simply be filled to a respective level, which may be predefined or given by the circumstances. It is also to be understood that A2, A3, A4 and AEW need not be fixed amounts but may vary between different instances of the respective phase during a treatment session.

As understood from the example in FIG. 6G, spent fluid is drawn from the peritoneal cavity and is pumped into the SF container 32′ of the first sub-system 32 in each drain phase. Whenever the SF container 32′ has been refilled with spent fluid during a drain phase, the system is configured to operate the FO unit 30, at a suitable time point, for example during a dwell phase subsequent to a fill phase after the drain phase, to extract water from the spent fluid to dilute the concentrate Ci, which is supplied by the second sub-system 36. The resulting diluted concentrate fluid is collected in the DCF container 372 of the third sub-system 37. During each fill phase, the diluted concentrate fluid is mixed with liquid water from the EW container 39 and optionally one or more further concentrates Cc.

In accordance with step 603 in FIG. 6B, the FO process is adjusted based on the estimated amount of available EW water and the water consumption rate, which corresponds to the production of final dialysis fluid, FDF. In one example, a patient shall receive 12 liters of FDF during a session including 6 dwell phases, hence 2 liters of FDF per dwell phase. Thus, for each fill phase before a dwell phase, 2 liters of FDF needs to be produced. In the context of FIG. 6B, it is assumed that the Ce concentrate is omitted and that the Ci concentrate is a pre-made liquid and is diluted 1:20. Thus, to produce 2 liters of dialysis fluid, 100 ml Ci concentrate is needed and 1900 ml of water. In total, about 11.40 liters of water is needed (1.90×6). When the first drain phase starts, there are 4 liters of liquid water in the EW container 39 (measured with the scale 378) and it is estimated that 1 liter of liquid water additionally will be extracted from the ambient air by the WSU 20 during the session. Hence, 5 liters of liquid water (EW) is available. Equally distributed over the fill phases, this means that ⅚ liters of liquid water is available for each fill phase. In accordance with the second embodiment (FIG. 3B), this liquid water may be added to the diluted concentrate fluid, DCF, downstream the FO process, to further dilute the DCF. In such a case, the FO process needs to extract the missing amount of water, 1.90 liters minus ⅚ liters, which is approximately 1.07 liters. The target value of water extraction from the spent fluid in the FO process may thus be set to at least 1.07 liters. In practice, the target value will be set with a margin to the missing amount of water, provided that the FO unit 30 does not have to be operated at maximum capacity to reach the target value. Based on, for example, tabular data for the Ci concentrate and the spent fluid, characteristic data for the FO unit, and the target value of water extraction, process parameters of the FO unit may be determined. For example, a concentration of the DCF to be reached may be determined and the process parameters controlled to achieve such concentration as measured with the first sensor 371 being, for example, a conductivity sensor. The remaining ⅚ liters of water is thereafter added to the DCF when the final dialysis fluid FDF is prepared.

While the foregoing description has focused on PD therapy, the method 600 in FIG. 6B is equally applicable to EC blood therapy. One difference between EC blood therapy and PD therapy is that the water consumption rate (WCR) is typically continuous during a session of EC blood therapy, compared to the intermittent water consumption and SF production of PD therapy. In EC blood therapy, there is thus a continuous flow of SF into the SF container, making it possible to more freely select the time for performing FO processing to produce DCF. The relaxed requirement for timing of FO processing may facilitate the implementation of the method 600 for use with EC blood therapy.

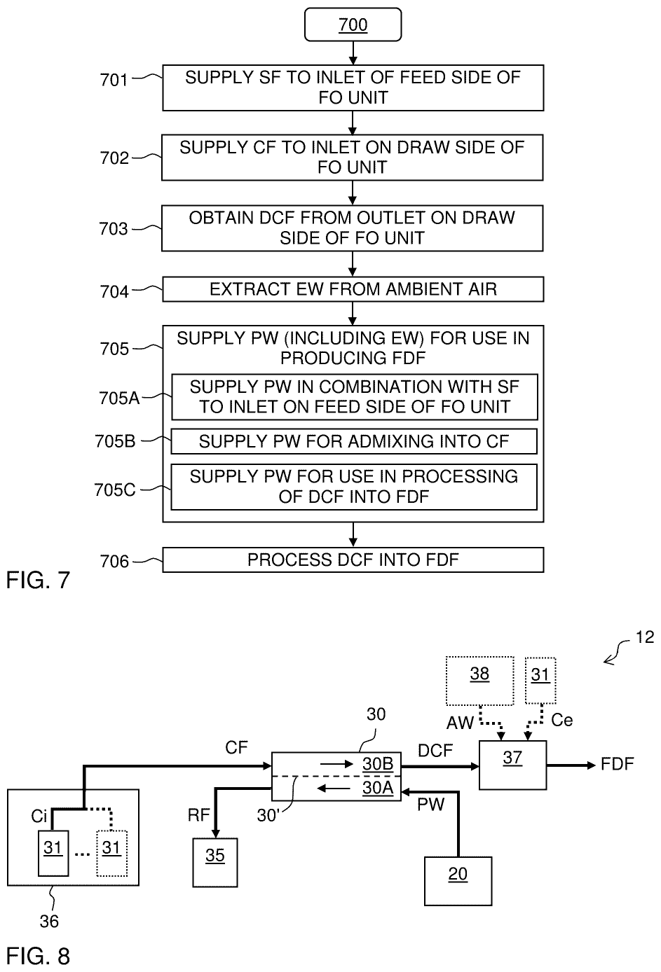

FIG. 7 is a flow chart of a method 700 of providing treatment fluid, which may be performed by the systems disclosed herein. In step 701, spent fluid (SF) is supplied to an inlet 30Ai on the feed side 30A of the FO unit 30. In step 702, the concentrate fluid (CF) is supplied to an inlet 30Bi on the draw side 30B of the FO unit 30 and is thereby diluted into a diluted concentrate fluid (DCF). In step 703, the diluted concentrate fluid (DCF) is obtained from an outlet 30Bo on the draw side 30B of the FO unit 30. In step 704, liquid water (EW) is extracted from ambient air. In step 705, process water (PW), which includes the liquid water (EW), is supplied for use in producing the final dialysis fluid (FDF). As shown in FIG. 7, step 705 comprises at least one of: a step 705A of supplying the process water (PW) in combination with the spent fluid (SF) to the inlet 30Ai on the feed side 30A of the FO unit 30, a step 705B of supplying the process water (PW) for admixing into the concentrate fluid (CF), or a step 705C of supplying the process water (PW) for use in step 706. In step 706, the diluted concentrate fluid (DCF) is processed into the final dialysis fluid (FDF).

FIG. 8 shows an example of another type of FPS 12, in which PW from the WSU 20 is supplied to the feed side 30A of the FO unit 30. The first sub-system 32 is replaced by the WSU 20, and the FPS 12 does not operate on SF. Thus, in contrast to the first to third embodiments, the water is extracted through the membrane 30′ from PW on the feed side 30A to CF on the draw side 30B, thereby generating DCF. Like in the first to third embodiments, the FPS 12 comprises a second sub-system 36 and a third sub-system 37, which may be configured in accordance with any examples given hereinabove with reference to the first to third embodiments. One technical advantage of the variant in FIG. 8 is that the FPS 12 is operable for any quality of PW, due to the inherent purification and/or sterilization performed by the membrane 30′. Although not shown in FIG. 8, the FPS 12 may be configured to circulate PW multiple passes through the feed side 30A to enable sufficient water extraction in the FO unit 30.

The FPS 12 in FIG. 8 is a system for producing dialysis fluid. The system comprises: a forward osmosis, FO, unit 30 comprising a feed side 30A and a draw side 30B separated by an FO membrane 30′, wherein the FO unit 30 is arranged to receive process water, PW, at an inlet on the feed side 30A and receive a concentrate fluid, CF, at an inlet on the draw side 30B, wherein the FO unit 30 is configured to transport water from the process water, PW, to the concentrate fluid, CF, through the FO membrane 30′ via an osmotic pressure gradient between the feed side 30A and the draw side 30B, thereby diluting the concentrate fluid, CF, into a diluted concentrate fluid, DCF. The system further comprises: a water supply unit 20 fluidly connected to provide the process water, PW, to the inlet on the feed side 30A of the FO unit 30, a second fluid sub-system 36 fluidly connected to provide the concentrate fluid, CF, to the inlet on the draw side 30B of the FO unit 30, and a third sub-system 37 fluidly connected to receive the diluted concentrate fluid, DCF, from an outlet on the draw side 30B of the FO unit 30, the third sub-system 37 being configured to process the diluted concentrate fluid, DCF, into a final dialysis fluid, FDF, wherein the water supply unit 20 is configured to extract liquid water, EW, from ambient air and provide the process water, PW, that includes the extracted liquid water, EW.

As noted, the systems described herein are operable to produce dialysis fluid or replacement fluid for EC blood therapy. In some embodiments, dialysis/replacement fluid for use in treatment of patients with chronic kidney disease (CKG) is generated by mixing a single concentrate with water at a dilution ratio of 10-50 by volume. In a non-limiting example, the single concentrate comprises lactate, sodium, potassium, calcium, magnesium, glucose and chloride. Such a concentrate is, for example, commercially available for the PureFlow SL system from NxStage. Alternatively, dialysis/replacement fluid may be generated by mixing two concentrates with water. For example, a base concentrate and an acid concentrate may be mixed with water at a dilution ratio of 10-50. Such concentrates are commercially available and well-known in the art. In a non-limiting example, the base concentrate comprises a buffer, for example bicarbonate, and the acid concentrate comprises sodium, potassium, calcium, magnesium, glucose, acetate and chloride. In some acid concentrates, acetate is replaced or supplemented by another acid, for example citric acid. In some embodiments, dialysis/replacement fluid for CRRT treatment of patients with acute kidney injury (AKI) is generated by mixing at least one concentrate with water. In a non-limiting example, such a dialysis/replacement fluid comprises bicarbonate, sodium, potassium, calcium, magnesium, phosphate, glucose, acetate and chloride. In one example, a base concentrate and an electrolyte concentrate may be mixed with water to form the dialysis/replacement fluid. For example, the base concentrate may be an alkaline hydrogen carbonate solution, and the electrolyte concentrate may be an acidic glucose-based electrolyte solution.

As noted, the systems described herein are also operable to produce dialysis fluid for PD therapy by mixing at least one concentrate with water. Example compositions of concentrates to be mixed with water are disclosed in US2018/0021501 and WO2017/193069, which are incorporated herein by reference. In one example, the one or more concentrates comprises ions and/or salts, such as lactate, acetate, citrate, bicarbonate, KCl, MgCL2, CaCl2, NaCl, and an osmotic agent. In any of the embodiments described herein, the osmotic agent may be, or include, glucose (or polyglucose), L-carnitine, glycerol, icodextrin, or any other suitable agent. For example, icodextrin is a glucose polymer preparation commonly used as osmotic agent in PD fluids. Alternative osmotic agents may be fructose, sorbitol, mannitol and xylitol. It is noted that glucose is also sometimes named as dextrose in the PD field. The term glucose is herewith intended to comprise dextrose.

While the subject of the present disclosure has been described in connection with what is presently considered to be the most practical embodiments, it is to be under-stood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results.

1-35. (canceled).

36. A system for producing dialysis fluid based on spent fluid, the system comprising:
a forward osmosis (FO) unit comprising a feed side and a draw side separated by an FO membrane, wherein the FO unit is arranged to receive the spent fluid at an inlet on the feed side and receive a concentrate fluid at an inlet on the draw side, wherein the FO unit is configured to transport water from the spent fluid to the concentrate fluid through the FO membrane via an osmotic pressure gradient between the feed side and the draw side, thereby diluting the concentrate fluid into a diluted concentrate fluid,
a first fluid sub-system fluidly connected to provide the spent fluid to the inlet on the feed side of the FO unit,
a second fluid sub-system fluidly connected to provide the concentrate fluid to the inlet on the draw side of the FO unit, and
a third fluid sub-system fluidly connected to receive the diluted concentrate fluid from an outlet on the draw side of the FO unit, the third fluid sub-system being configured to process the diluted concentrate fluid into a final dialysis fluid,
wherein the system is arranged to provide the final dialysis fluid to a therapy system, which is configured for performing dialysis therapy in treatment sessions, and receive the spent fluid from the therapy system, the spent fluid comprising spent dialysis fluid generated by the therapy system as a result of the dialysis therapy,
the system further comprises:
a water supply unit which is configured to extract liquid water from ambient air and provide process water that includes the extracted liquid water,
wherein the water supply unit is fluidly connected to provide the process water to at least one of (i) the first fluid sub-system for combination with the spent fluid, (ii) the second fluid sub-system for admixing into the concentrate fluid, or (iii) the third fluid sub-system for use in processing the diluted concentrate fluid into the final dialysis fluid, and
a control arrangement, which is configured to jointly operate the water supply unit and the FO unit to achieve a target production rate of the final dialysis fluid, wherein the target production rate is adapted to a consumption rate of the final dialysis fluid during an upcoming treatment session of the dialysis therapy,
wherein the control arrangement is configured to estimate a water consumption rate for the upcoming treatment session, and determine an estimated amount of the process water that is produced by the water supply unit between treatment sessions, and optionally during the upcoming treatment session, and configure the FO unit for operation during the upcoming treatment session in dependence of the estimated water consumption rate and the estimated amount of the process water.

37. The system of claim 36, wherein the water supply unit comprises a desiccant, which is arranged to adsorb and/or absorb moisture from an incoming stream of ambient air and which is processed by the water supply unit to extract the liquid water from the desiccant.

38. The system of claim 37, wherein the desiccant is configured to have a high selectivity towards water.

39. The system of claim 36, wherein the liquid water that is extracted from the ambient air has conductivity of less than 10 μS/cm, and preferably less than 5 μS/cm or 1 μS/cm.

40. The system of claim 36, wherein the water supply unit comprises a cooling element, which is configured to cool an incoming stream of ambient air to extract the liquid water from the incoming stream of ambient air by condensation.

41. The system of claim 36, wherein the process water consists of the extracted liquid water.

42. The system of claim 36, wherein the FO unit has a maximum water extraction capacity, and wherein the control arrangement is configured, in view of the estimated amount of the process water, to operate the FO unit with a water extraction capacity below the maximum water extraction capacity during the upcoming treatment session.

43. The system of claim 42, wherein the control arrangement is configured to operate the water supply unit to produce the estimated amount of the process water so that the FO unit is operable at a fraction, α, of the maximum water extraction capacity, wherein α≤0.95 and preferably α≤0.9.

44. The system of claim 36, wherein the control arrangement is configured to operate the FO unit to produce less than 90% of an estimated total consumption of the process water during the upcoming treatment session.

45. The system of claim 36, wherein the control arrangement is configured to operate the water supply unit to maximize extraction of the liquid water from the ambient air, at least between treatment sessions, while maintaining a humidity of the ambient air above a humidity limit.

46. The system of claim 36, wherein the process water that is produced by the water supply unit is stored in a PW container, which is fluidly connected to at least one of the first fluid sub-system, the second fluid sub-system or the third fluid sub-system.

47. The system of claim 46, wherein each treatment session comprises a series of fluid exchange cycles, wherein each of the fluid exchange cycles comprises a fill phase, in which a first amount of the final dialysis fluid is supplied to a peritoneal cavity of a patient, a dwell phase, in which the final dialysis fluid resides in the peritoneal cavity, and a drain phase, in which a second amount of the spent fluid is withdrawn from the peritoneal cavity, and wherein the PW container is fluidly connected to the third fluid sub-system to provide the processing water for use in processing the diluted concentrate fluid into the final dialysis fluid.

48. The system of claim 47, wherein the control arrangement is configured to operate the FO unit to produce a third amount of the diluted concentrate fluid from the second amount of the spent fluid withdrawn in a respective drain phase, and operate the third fluid sub-system to generate the first amount of the final dialysis fluid for use in a fill phase, which is subsequent to the respective drain phase, based on the third amount of the diluted concentrate fluid and a supplementary amount of the process water in the PW container.

49. The system of claim 48, wherein the control arrangement is configured to operate the water supply unit, at least between treatment sessions, to generate and accumulate process water in the PW container, wherein the control arrangement is configured to set a target value for the third amount based on a fourth amount of process water in the PW container at start of the upcoming treatment session.

50. The system of claim 36, wherein the water supply unit is fluidly connected to provide the process water to at least one of the second fluid sub-system or the third fluid sub-system, the system further comprising at least one sterilization unit, which is arranged to sterilize at least one of the process water, the concentrate fluid, the diluted concentrate fluid or the final dialysis fluid.

51. The system of claim 36, which comprises a sensor arrangement, which is configured to generate sensor data representative of spatial structures around the water supply unit, and which is configured to process the sensor data to estimate an available volume of ambient air and operate the water supply unit based on the available volume of ambient air.

52. The system of claim 36, wherein the third fluid sub-system comprises at least one mixing section for mixing the diluted concentrate fluid with process water and/or at least one dialysis concentrate.

53. The system of claim 52, wherein the third fluid sub-system is configured to first mix the diluted concentrate fluid with the process water to generate a fluid mixture and then mix the fluid mixture with the at least one dialysis concentrate.

54. The system of claim 52, wherein the third fluid sub-system comprises a first sensor for measuring a first concentration of the diluted concentrate fluid and second sensor for measuring a second concentration of the final dialysis fluid, and wherein the system is operable to control, based on the first and second concentrations, a flow rate of the diluted concentrate fluid, and one or more flow rates of the process water and/or the at least one dialysis concentrate.

55. A method of producing dialysis fluid based on spent fluid, the method comprising:
supplying spent fluid to an inlet on a feed side of a forward osmosis (FO) unit;
supplying a concentrate fluid to an inlet on a draw side of the FO unit, the draw side being separated from the feed side by an FO membrane, the FO unit being configured to transport water from the spent fluid to the concentrate fluid through the FO membrane via an osmotic pressure gradient between the feed side and the draw side, thereby diluting the concentrate fluid into a diluted concentrate fluid;
obtaining the diluted concentrate fluid from an outlet on the draw side of the FO unit;
processing the diluted concentrate fluid into a final dialysis fluid;
providing the final dialysis fluid to a therapy system, which is configured for performing dialysis therapy in treatment sessions;
receiving the spent fluid from the therapy system, the spent fluid comprising spent dialysis fluid generated by the therapy system as a result of the dialysis therapy;
extracting liquid water from ambient air by a water supply unit;
supplying process water, which includes the extracted liquid water, for use in producing the final dialysis fluid, wherein the supplying the process water comprises at least one of (i) supplying the process water in combination with the spent fluid to the inlet on the feed side of the FO unit, (ii) supplying the process water for admixing into the concentrate fluid, or (iii) supplying the process water for use in the processing the diluted concentrate fluid into the final dialysis fluid; and
jointly operating the water supply unit and the FO unit to achieve a target production rate of the final dialysis fluid, wherein the target production rate is adapted to a consumption rate of the final dialysis fluid during an upcoming treatment session of the dialysis therapy,
wherein the jointly operating the water supply comprises:
estimating a water consumption rate for the upcoming treatment session,
determining an estimated amount of the process water that is produced by the water supply unit between treatment sessions, and optionally during the upcoming treatment session, and
configuring the FO unit for operation during the upcoming treatment session in dependence of the estimated water consumption rate and the estimated amount of the process water.
